SEQUENCE LISTING (10 sequences)

SEQ ID NO: 1 - protein, 516 aa, Homo sapiens
MEPAVSLAVCALLFLLWVRLKGLEFVLIHQRWVFVCLFLLPLSLIFDIYYYVRAWVVFKLSSAPRLHEQRVRDIQKQVREWKEQGSKTFMCTGRPGWLTVSLRVGKYKKTHKNIMINLMDILEVDTKKQIVRVEPLVTMGQVTALLTSIGWTLPVLPELDDLTVGGLIMGTGIESSSHKYGLFQHICTAYELVLADGSFVRCTPSENSDLFYAVPWSCGTLGFLVAAEIRIIPAKKYVKLRFEPVRGLEAICAKFTHESQRQENHFVEGLLYSLDEAVIMTGVMTDEAEPSKLNSIGNYYKPWFFKHVENYLKTNREGLEYIPLRHYYHRHTRSIFWELQDIIPFGNNPIFRYLFGWMVPPKISLLKLTQGETLRKLYEQHHVVQDMLVPMKCLQQALHTFQNDIHVYPIWLCPFILPSQPGLVHPKGNEAELYIDIGAYGEPRVKHFEARSCMRQLEKFVRSVHGFQMLYADCYMNREEFWEMFDGSLYHKLREKLGCQDAFPEVYDKICKAARH

SEQ ID NO: 2 - DNA, 4248 bp, Homo sapiens
cccgggctgtgggctacaggcgcagagcgggccaggcgcggagctggcggcagtgacaggaggcgcgaacccgcagcgcttaccgcgcggcgccgcaccatggagcccgccgtgtcgctggccgtgtgcgcgctgctcttcctgctgtgggtgcgcctgaaggggctggagttcgtgctcatccaccagcgctgggtgttcgtgtgcctcttcctcctgccgctctcgcttatcttcgatatctactactacgtgcgcgcctgggtggtgttcaagctcagcagcgctccgcgcctgcacgagcagcgcgtgcgggacatccagaagcaggtgcgggaatggaaggagcagggtagcaagaccttcatgtgcacggggcgccctggctggctcactgtctcactacgtgtcgggaagtacaagaagacacacaaaaacatcatgatcaacctgatggacattctggaagtggacaccaagaaacagattgtccgtgtggagcccttggtgaccatgggccaggtgactgccctgctgacctccattggctggactctccccgtgttgcctgagcttgatgacctcacagtggggggcttgatcatgggcacaggcatcgagtcatcatcccacaagtacggcctgttccaacacatctgcactgcttacgagctggtcctggctgatggcagctttgtgcgatgcactccgtccgaaaactcagacctgttctatgccgtaccctggtcctgtgggacgctgggtttcctggtggccgctgagatccgcatcatccctgccaagaagtacgtcaagctgcgtttcgagccagtgcggggcctggaggctatctgtgccaagttcacccacgagtcccagcggcaggagaaccacttcgtggaagggctgctctactccctggatgaggctgtcattatgacaggggtcatgacagatgaggcagagcccagcaagctgaatagcattggcaattactacaagccgtggttctttaagcatgtggagaactatctgaagacaaaccgagagggcctggagtacattcccttgagacactactaccaccgccacacgcgcagcatcttctgggagctccaggacatcatcccctttggcaacaaccccatcttccgctacctctttggctggatggtgcctcccaagatctccctcctgaagctgacccagggtgagaccctgcgcaagctgtacgagcagcaccacgtggtgcaggacatgctggtgcccatgaagtgcctgcagcaggccctgcacaccttccaaaacgacatccacgtctaccccatctggctgtgtccgttcatcctgcccagccagccaggcctagtgcaccccaaaggaaatgaggcagagctctacatcgacattggagcatatggggagccgcgtgtgaaacactttgaagccaggtcctgcatgaggcagctggagaagtttgtccgcagcgtgcatggcttccagatgctgtatgccgactgctacatgaaccgggaggagttctgggagatgtttgatggctccttgtaccacaagctgcgagagaagctgggttgccaggacgccttccccgaggtgtacgacaagatctgcaaggccgccaggcactgagctggagcccgcctggagagacagacacgtgtgagtggtcaggcatcttcccttcactcaagcttggctgctttcctagatccacactttcaaagagaaacccctccagaactcccaccctgacagcccaacaccaccttcctcctggcttccagggggcagcccagtggaatggaaagaatgtgggatttggagtcagacaagcctgagtccagttccccgtttagaactcattagctgtgtgactctgggtgagtcccttaacccctctgagcccgggtctcttcattagttgaaagggatagtaatacctacttgcaggttgttgtcatctgagttgagcactggtcacattgaaggtgctgggtaagtggtagctcttgttgcttcccgttcagcgtcacatctgcagtggagcctgaaaaggctccacattaggtcacctgtgcacagccatggctggaatgatgaaggggatacgctggagttgccctgccatcgcctccatcagccagacgaggtcctcacaggagaaggacagctcttccccaccctgggatctcaggagggcagccacggagtggggaggccccagatgcgctgtgccaaagccaggtccgaggccaaagttctccctgccatccttggtgccgtcctgccccttcctccttcatgcctgggcctgcaggcccaccccagccaccactgagtccactcggagtgccctgtgttcctggagaaggcattccagggttgaatcttgtcccagcctcagcctgggacacctaggtggagagagtggtctccgctctgaattggatccaggggacctgggctcattcttcttggctcaccaaccctgcaggcctcatctttcccaaaacccactttgtcttggtgggagtgggtccgcgctgctctgcagcaggggctggggagtggacagcatcaggtgggaaagtggagtccaccctcatgtttctgtaggattctcaccgtggggctggaagaaaagagcatcgacttgatttctccaaccactcatccctctttttctttcttccaccactccccaccccagctgtagttaatttcagtgccttacaaatcctaagctcagagaaagttccatttccgttccagagggaagggaacctccctaggtccttccctggcttgttataacgcaaagcttggttgtttatgcaactctatcttaagaactgcccagcctcagctgaaaacccgaatctgagaaggaattgcgtcatgtaagggaagctggaattaagggagctgagccagtcatggttgtggcgtgtgagtcaggagacctaggtttcagcccctctctactgtcagcgagctgtgcaacgtgggcaagtcattgtcctctgagctgcagtttcctcatctgtcacatcgctacagacaagacctccctggaacccttctgattgtcttagacactgtggttgcaaaacccacggaaagcctcatttgtgtggaaagtcagaggaaaaatgatccagtggacacttggggattatctgtcattcaagatccttccttcaaccccaaggccagctcccatctcatttccagaaaggctcatacctggcttgcagggaagcatctgtcttgtcattccaggtgccagaatcctctcagagtcattgaagggtgttcacccatcccacccaaggcttggcacactgccagtgtcttagcagggtcttgtgagggctgggggcatccaggcactcagaaggcaaaggaaccaccctacccatttggcctctggagggggcagaagaaagaaagaaacctcatcctatattttacaaagcatgtgaattctggcattagctctcataggagacccatgtgcttccttgctcagtgcaaaactgatgattctacttgctgtagatgaatggttaacacgagctagttaaacagtgccattgttttgccagtgaagcctccaaccctaagccactgggacggtggccagagatgccagcagcctctgtcgcccttagtcatataaccaaaatccagaccttatccacaacccggggcttggaaaggaaggtattttggaatcacaccctccggttatgttgctccagtaaaatcttgcctggaaagaggcagtcttcttagcatggtgagctgagttcatggcttttttttgtagccagtcctgtccctggccatccatgtgatggttttggatggagttaaacttgatgccagtgggcagtgcatgtggaaagtatcagagtaagcctctcccctccagagccctgagtttcttggctgcatgaaggttttctttagaatcagaattgtagccagtttctttggccagaaggatgaatacttggatattactgaaagggaggggtggagatgggtgtggcagtgtatggtgtgtgatttttattttcttctttggtcatgggggccaaggagaaaggcatgaatcttccctgtcaggctcttacagccacaggcactgtgtctactgtctggaagacatgtccccgtggctgtggggccgctgcttctgtttaaataaaagtggcctgg

SEQ ID NO: 3 - DNA, 4187 bp, Homo sapiens
ggcgcgaacccgcagcgcttaccgcgcggcgccgcaccatggagcccgccgtgtcgctggccgtgtgcgcgctgctcttcctgctgtgggtgcgcctgaaggggctggagttcgtgctcatccaccagcgctgggtgttcgtgtgcctcttcctcctgccgctctcgcttatcttcgatatctactactacgtgcgcgcctgggtggtgttcaagctcagcagcgctccgcgcctgcacgagcagcgcgtgcgggacatccagaagcaggtgcgggaatggaaggagcagggtagcaagaccttcatgtgcacggggcgccctggctggctcactgtctcactacgtgtcgggaagtacaagaagacacacaaaaacatcatgatcaacctgatggacattctggaagtggacaccaagaaacagattgtccgtgtggagcccttggtgaccatgggccaggtgactgccctgctgacctccattggctggactctccccgtgttgcctgagcttgatgacctcacagtggggggcttgatcatgggcacaggcatcgagtcatcatcccacaagtacggcctgttccaacacatctgcactgcttacgagctggtcctggctgatggcagctttgtgcgatgcactccgtccgaaaactcagacctgttctatgccgtaccctggtcctgtgggacgctgggtttcctggtggccgctgagatccgcatcatccctgccaagaagtacgtcaagctgcgtttcgagccagtgcggggcctggaggctatctgtgccaagttcacccacgagtcccagcggcaggagaaccacttcgtggaagggctgctctactccctggatgaggctgtcattatgacaggggtcatgacagatgaggcagagcccagcaagctgaatagcattggcaattactacaagccgtggttctttaagcatgtggagaactatctgaagacaaaccgagagggcctggagtacattcccttgagacactactaccaccgccacacgcgcagcatcttctgggagctccaggacatcatcccctttggcaacaaccccatcttccgctacctctttggctggatggtgcctcccaagatctccctcctgaagctgacccagggtgagaccctgcgcaagctgtacgagcagcaccacgtggtgcaggacatgctggtgcccatgaagtgcctgcagcaggccctgcacaccttccaaaacgacatccacgtctaccccatctggctgtgtccgttcatcctgcccagccagccaggcctagtgcaccccaaaggaaatgaggcagagctctacatcgacattggagcatatggggagccgcgtgtgaaacactttgaagccaggtcctgcatgaggcagctggagaagtttgtccgcagcgtgcatggcttccagatgctgtatgccgactgctacatgaaccgggaggagttctgggagatgtttgatggctccttgtaccacaagctgcgagagaagctgggttgccaggacgccttccccgaggtgtacgacaagatctgcaaggccgccaggcactgagctggagcccgcctggagagacagacacgtgtgagtggtcaggcatcttcccttcactcaagcttggctgctttcctagatccacactttcaaagagaaacccctccagaactcccaccctgacagcccaacaccaccttcctcctggcttccagggggcagcccagtggaatggaaagaatgtgggatttggagtcagacaagcctgagtccagttccccgtttagaactcattagctgtgtgactctgggtgagtcccttaacccctctgagcccgggtctcttcattagttgaaagggatagtaatacctacttgcaggttgttgtcatctgagttgagcactggtcacattgaaggtgctgggtaagtggtagctcttgttgcttcccgttcagcgtcacatctgcagtggagcctgaaaaggctccacattaggtcacctgtgcacagccatggctggaatgatgaaggggatacgctggagttgccctgccatcgcctccatcagccagacgaggtcctcacaggagaaggacagctcttccccaccctgggatctcaggagggcagccacggagtggggaggccccagatgcgctgtgccaaagccaggtccgaggccaaagttctccctgccatccttggtgccgtcctgccccttcctccttcatgcctgggcctgcaggcccaccccagccaccactgagtccactcggagtgccctgtgttcctggagaaggcattccagggttgaatcttgtcccagcctcagcctgggacacctaggtggagagagtggtctccgctctgaattggatccaggggacctgggctcattcttcttggctcaccaaccctgcaggcctcatctttcccaaaacccactttgtcttggtgggagtgggtccgcgctgctctgcagcaggggctggggagtggacagcatcaggtgggaaagtggagtccaccctcatgtttctgtaggattctcaccgtggggctggaagaaaagagcatcgacttgatttctccaaccactcatccctctttttctttcttccaccactccccaccccagctgtagttaatttcagtgccttacaaatcctaagctcagagaaagttccatttccgttccagagggaagggaacctccctaggtccttccctggcttgttataacgcaaagcttggttgtttatgcaactctatcttaagaactgcccagcctcagctgaaaacccgaatctgagaaggaattgcgtcatgtaagggaagctggaattaagggagctgagccagtcatggttgtggcgtgtgagtcaggagacctaggtttcagcccctctctactgtcagcgagctgtgcaacgtgggcaagtcattgtcctctgagctgcagtttcctcatctgtcacatcgctacagacaagacctccctggaacccttctgattgtcttagacactgtggttgcaaaacccacggaaagcctcatttgtgtggaaagtcagaggaaaaatgatccagtggacacttggggattatctgtcattcaagatccttccttcaaccccaaggccagctcccatctcatttccagaaaggctcatacctggcttgcagggaagcatctgtcttgtcattccaggtgccagaatcctctcagagtcattgaagggtgttcacccatcccacccaaggcttggcacactgccagtgtcttagcagggtcttgtgagggctgggggcatccaggcactcagaaggcaaaggaaccaccctacccatttggcctctggagggggcagaagaaagaaagaaacctcatcctatattttacaaagcatgtgaattctggcattagctctcataggagacccatgtgcttccttgctcagtgcaaaactgatgattctacttgctgtagatgaatggttaacacgagctagttaaacagtgccattgttttgccagtgaagcctccaaccctaagccactgggacggtggccagagatgccagcagcctctgtcgcccttagtcatataaccaaaatccagaccttatccacaacccggggcttggaaaggaaggtattttggaatcacaccctccggttatgttgctccagtaaaatcttgcctggaaagaggcagtcttcttagcatggtgagctgagttcatggcttttttttgtagccagtcctgtccctggccatccatgtgatggttttggatggagttaaacttgatgccagtgggcagtgcatgtggaaagtatcagagtaagcctctcccctccagagccctgagtttcttggctgcatgaaggttttctttagaatcagaattgtagccagtttctttggccagaaggatgaatacttggatattactgaaagggaggggtggagatgggtgtggcagtgtatggtgtgtgatttttattttcttctttggtcatgggggccaaggagaaaggcatgaatcttccctgtcaggctcttacagccacaggcactgtgtctactgtctggaagacatgtccccgtggctgtggggccgctgcttctgtttaaataaaagtggcctgg

SEQ ID NO: 4 - DNA, 4186 bp, Homo sapiens
ggcgcgaacccgcagcgcttaccgcgcggcgccgcaccatggagcccgccgtgtcgctggccgtgtgcgcgctgctcttcctgctgtgggtgcgcctgaaggggctggagttcgtgctcatccaccagcgctgggtgttcgtgtgcctcttcctcctgccgctctcgcttatcttcgatatctactactacgtgcgcgcctgggtggtgttcaagctcagcagcgctccgcgcctgcacgagcagcgcgtgcgggacatccagaagcaggtgcgggaatggaaggagcagggtagcaagaccttcatgtgcacggggcgccctggctggctcactgtctcactacgtgtcgggaagtacaagaagacacacaaaaacatcatgatcaacctgatggacattctggaagtggacaccaagaaacagattgtccgtgtggagcccttggtgaccatgggccaggtgactgccctgctgacctccattggctggactctccccgtgttgcctgagcttgatgacctcacagtggggggcttgatcatgggcacaggcatcgagtcatcatcccacaagtacggcctgttccaacacatctgcactgcttacgagctggtcctggctgatggcagctttgtgcgatgcactccgtccgaaaactcagacctgttctatgccgtaccctggtcctgtgggacgctgggtttcctggtggccgctgagatccgcatcatccctgccaagaagtacgtcaagctgcgtttcgagccagtgcggggcctggaggctatctgtgccaagttcacccacgagtcccagcggcaggagaaccacttcgtggaagggctgctctactccctggatgaggctgtcattatgacaggggtcatgacagatgaggcagagcccagcaagctgaatagcattggcaattactacaagccgtggttctttaagcatgtggagaactatctgaagacaaaccgagagggcctggagtacattcccttgagacactactaccaccgccacacgcgcagcatcttctgggagctccaggacatcatcccctttggcaacaaccccatcttccgctacctctttggctggatggtgcctcccaagatctccctcctgaagctgacccagggtgagaccctgcgcaagtgtacgagcagcaccacgtggtgcaggacatgctggtgcccatgaagtgcctgcagcaggccctgcacaccttccaaaacgacatccacgtctaccccatctggctgtgtccgttcatcctgcccagccagccaggcctagtgcaccccaaaggaaatgaggcagagctctacatcgacattggagcatatggggagccgcgtgtgaaacactttgaagccaggtcctgcatgaggcagctggagaagtttgtccgcagcgtgcatggcttccagatgctgtatgccgactgctacatgaaccgggaggagttctgggagatgtttgatggctccttgtaccacaagctgcgagagaagctgggttgccaggacgccttccccgaggtgtacgacaagatctgcaaggccgccaggcactgagctggagcccgcctggagagacagacacgtgtgagtggtcaggcatcttcccttcactcaagcttggctgctttcctagatccacactttcaaagagaaacccctccagaactcccaccctgacagcccaacaccaccttcctcctggcttccagggggcagcccagtggaatggaaagaatgtgggatttggagtcagacaagcctgagtccagttccccgtttagaactcattagctgtgtgactctgggtgagtcccttaacccctctgagcccgggtctcttcattagttgaaagggatagtaatacctacttgcaggttgttgtcatctgagttgagcactggtcacattgaaggtgctgggtaagtggtagctcttgttgcttcccgttcagcgtcacatctgcagtggagcctgaaaaggctccacattaggtcacctgtgcacagccatggctggaatgatgaaggggatacgctggagttgccctgccatcgcctccatcagccagacgaggtcctcacaggagaaggacagctcttccccaccctgggatctcaggagggcagccacggagtggggaggccccagatgcgctgtgccaaagccaggtccgaggccaaagttctccctgccatccttggtgccgtcctgccccttcctccttcatgcctgggcctgcaggcccaccccagccaccactgagtccactcggagtgccctgtgttcctggagaaggcattccagggttgaatcttgtcccagcctcagcctgggacacctaggtggagagagtggtctccgctctgaattggatccaggggacctgggctcattcttcttggctcaccaaccctgcaggcctcatctttcccaaaacccactttgtcttggtgggagtgggtccgcgctgctctgcagcaggggctggggagtggacagcatcaggtgggaaagtggagtccaccctcatgtttctgtaggattctcaccgtggggctggaagaaaagagcatcgacttgatttctccaaccactcatccctctttttctttcttccaccactccccaccccagctgtagttaatttcagtgccttacaaatcctaagctcagagaaagttccatttccgttccagagggaagggaacctccctaggtccttccctggcttgttataacgcaaagcttggttgtttatgcaactctatcttaagaactgcccagcctcagctgaaaacccgaatctgagaaggaattgcgtcatgtaagggaagctggaattaagggagctgagccagtcatggttgtggcgtgtgagtcaggagacctaggtttcagcccctctctactgtcagcgagctgtgcaacgtgggcaagtcattgtcctctgagctgcagtttcctcatctgtcacatcgctacagacaagacctccctggaacccttctgattgtcttagacactgtggttgcaaaacccacggaaagcctcatttgtgtggaaagtcagaggaaaaatgatccagtggacacttggggattatctgtcattcaagatccttccttcaaccccaaggccagctcccatctcatttccagaaaggctcatacctggcttgcagggaagcatctgtcttgtcattccaggtgccagaatcctctcagagtcattgaagggtgttcacccatcccacccaaggcttggcacactgccagtgtcttagcagggtcttgtgagggctgggggcatccaggcactcagaaggcaaaggaaccaccctacccatttggcctctggagggggcagaagaaagaaagaaacctcatcctatattttacaaagcatgtgaattctggcattagctctcataggagacccatgtgcttccttgctcagtgcaaaactgatgattctacttgctgtagatgaatggttaacacgagctagttaaacagtgccattgttttgccagtgaagcctccaaccctaagccactgggacggtggccagagatgccagcagcctctgtcgcccttagtcatataaccaaaatccagaccttatccacaacccggggcttggaaaggaaggtattttggaatcacaccctccggttatgttgctccagtaaaatcttgcctggaaagaggcagtcttcttagcatggtgagctgagttcatggcttttttttgtagccagtcctgtccctggccatccatgtgatggttttggatggagttaaacttgatgccagtgggcagtgcatgtggaaagtatcagagtaagcctctcccctccagagccctgagtttcttggctgcatgaaggttttctttagaatcagaattgtagccagtttctttggccagaaggatgaatacttggatattactgaaagggaggggtggagatgggtgtggcagtgtatggtgtgtgatttttattttcttctttggtcatgggggccaaggagaaaggcatgaatcttccctgtcaggctcttacagccacaggcactgtgtctactgtctggaagacatgtccccgtggctgtggggccgctgcttctgtttaaataaaagtggcctgg

SEQ ID NO: 5 - DNA, 24 bp, Artificial Sequence
  Description of Artificial Sequence Primer
gcgcttaccgcgcggcgccgcacc

SEQ ID NO: 6 - DNA, 24 bp, Artificial Sequence
  Description of Artificial Sequence Primer
gaccagggtacggcatagaacagg

SEQ ID NO: 7 - DNA, 24 bp, Artificial Sequence
  Description of Artificial Sequence Primer
agaagtacgtcaagctgcgtttcg

SEQ ID NO: 8 - DNA, 24 bp, Artificial Sequence
  Description of Artificial Sequence Primer
ttctctttgaaagtgtggatctag

SEQ ID NO: 9 - DNA, 18 bp, Artificial Sequence
  Description of Artificial Sequence Primer
tgccgaagcttggagctt

SEQ ID NO: 10 - DNA, 18 bp, Artificial Sequence
  Description of Artificial Sequence Primer
tgccgaagctttggtcat

(19) United States
(12) Patent Application Publication
(10) Pub. No.: US 2008/0261302 A1
(43) Pub. Date: Oct. 23, 2008

(54) Methods of diagnosing or treating neurological diseases and cell degeneration

(75) Inventors: Roger Nitsch, Zollikon (CH); Isabell Greeve, Hamburg (DE)

Correspondence Address: JACOBSON HOLMAN PLLC, 400 SEVENTH STREET N.W., SUITE 600, WASHINGTON, DC, 20004, US

(73) Assignee: Evotec NeuroSciences GmbH.

(21) Appl. No.: 12/000,307

(22) Filed: Dec. 11, 2007

Related U.S. Application Data
(63) Continuation of application No. 10/910,772, filed on Aug. 4, 2004, now abandoned.
Application No. 10/910,772 is a continuation of application No. 09/831,754, filed on Oct. 15, 2001, now abandoned.

(30) Foreign Application Priority Data
Nov. 12, 1998 (EP) 98 121 478.6

(51) Int. Cl. C12N 5/06; C12N 15/11; C12N 15/00; C12N 1/14; C12N 1/20

(52) U.S. Cl. 435/348; 536/23.1; 435/320.1; 435/252.3; 435/325; 435/255.1

(57) ABSTRACT

The invention discloses an isolated nucleic acid molecule encoding a protein molecule, the function of which is to protect cells against degeneration and/or cell death, wherein the amino acid sequence of the protein comprises the sequence shown in SEQ ID NO. 2 or a functional variant thereof.

Cell death is a common feature occurring in two distinct forms in nature. Necrosis results from physical or chemical insult while apoptosis or programmed cell death results from a self-destruction program within the cell in response to internal and external stimuli. Latter process is a gene-directed form of cell death that is essential for normal development and maintenance of multicellular organisms. Recent work has clearly demonstrated that dysregulation of apoptosis may underlie the pathogenesis of a variety of diseases. Apoptosis has been reported to occur in conditions characterized by ischaemia, e.g. myocardial infarction and stroke. It has been implicated in a number of liver disorders including obstructive jaundice. Hepatic damage due to toxins and drugs is also associated with apoptosis in hepatocytes. Apoptosis has also been identified as a key phenomenon in some diseases of the kidney, i.e. polycistic kidney, as well as in disorders of the pancreas like alcohol-induced pancreatitis and diabetes. AIDS and neurodegenerative disorders like Alzheimer's and Parkinson's disease represent the most widely studied group of disorders where an excess of apoptosis has been implicated. Amyotrophic lateral sclerosis, retinitis pigmentosa, epilepsy and alcoholic brain damage are other neurological disorders in which apoptosis has been implicated.

Neurological diseases are widely spread within a population and have a strong impact not only on patients' life but also on society as such. Therefore, there is a strong need to elucidate the causes and the underlying pathogenesis of such neurological diseases. Among such neurological diseases, Alzheimer's disease (AD) has a predominant position. Alzheimer's disease, first described by the Bavarian psychiatrist Alois Alzheimer in 1907, is a progressive neurological disorder which begins with short term memory loss and proceeds to loss of cognitive functions, disorientation, impairment of judgement and reasoning and, ultimately, dementia. It is the most common cause of dementia. AD has been estimated to afflict 5 to 11 percent of the population over age 65 and as much as 47 percent of the population over age 85. Moreover, as adults, born during the population boom of the 1940's and 1950's, approach the age when AD becomes more prevalent, the control and treatment of AD will become an even more significant health care problem. Familial forms of AD are genetically heterogeneous, but most with early onset are linked to mutations in the presenilin genes PSEN1 and PSEN2, as well as to mutations of the amyloid precursor gene APP. The majority of AD patients have no obvious family history and are classified as sporadic AD. The neuropathology of AD is characterized by a substantial loss of neurons and synapses, and by the formation in brain of amyloid plaques and neurofibrillary tangles. Amyloid plaques are evenly distributed throughout the neocortex and the hippocampus, whereas neurodegeneration occurs predominantly in the inferior temporal lobes, the entorhinal cortex, and the hippocampus. Similar neurons in the frontal, parietal, and occipital lobes are largely preserved from degeneration even in severe end-stage AD. These observations indicate selective vulnerability of specific population of neurons. Factors that determine selective vulnerability of neurons in AD brains are unknown.

To elucidate the causes of cell degeneration and cell death is a general aim of the present invention. More specifically, the present invention aims at elucidating the causes and the underlying pathogenesis of neurological diseases, in particular Alzheimer's disease it is therefore an object of the present invention to provide an insight into the pathogenesis of neurological diseases and to provide methods and materials which are suited for diagnosis and treatment of said diseases, cell degeneration and cell death.

The invention features an isolated nucleic acid molecule encoding a protein molecule whose amino acid sequence comprises the sequence shown in SEQ ID NO. 1 as well as the protein molecule according to SEQ ID NO.1. Hereinafter, the protein molecule of SEQ ID NO. 1 is denoted “SELADIN-1”. One function of SELADIN-1 is to protect cells against degeneration and cell death. In particular, cells of the nerve system, muscular system, prostate, stomach, testis, ovary, adrenal glands, mammary glands, liver, spleen, lung, trachea or placenta are protected against degeneration and/or cell death. Therefore, the present invention also features functional variants of SELADIN-1 which might have a modification of the given primary structure of SELADIN-1, but whose essential biological function remains unaffected. “Variants” of a protein molecule shown in SEQ ID NO.1 include for example proteins with conservative amino acid substitutions in highly conservative regions. For example, isoleucine, valine and leucine can each be substituted for one another. Aspartate and glutamate can be substituted for each other. Glutamine and asparagine can be substituted for each other. Serine and threonine can be substituted for each other. Amino acid substitutions in less conservative regions include e.g.: Isoleucine, valine and leucine can each be substituted for one another. Aspartate and glutamate can be substituted for each other. Glutamine and asparagine can be substituted for each other. Serine and threonine can be substituted for each other. Glycine and alanine can be substituted for each other. Alanine and valine can be substituted for each other. Methionine can be substituted for each of leucine, isoleucine or valine, and vice versa. Lysine and arginine can be substituted for each other. One of aspartate and glutamate can be substituted for one of arginine or lysine, and vice versa. Histidine can be substituted for arginine or lysine, and vice versa. Glutamine and glutamate can be substituted for each other. Asparagine and aspartate can be substituted for each other. Other examples of protein modifications include glycosilation and further posttranslational modifications. The invention also features the nucleic acid molecules encoding such functional variants of the protein molecule of SEQ ID NO. 1. Nucleic acid molecules can be DNA molecules, such as genomic DNA molecules or cDNA molecules, or RNA molecules, such as mRNA molecules. In particular, said nucleic acid molecule can be a cDNA molecule comprising a nucleotide sequence of SEQ ID NO. 2. The invention also features an isolated DNA molecule capable of hybridizing with the complement of the cDNA described in SEQ ID NO. 2 under stringent conditions. Examples for stringent conditions include (i) 0.2×SSC (standard saline citrate) and 0.1% SDS at 60° C. and (ii) 50% formamide, 4×SSC, 50 mM HEPES, pH 7.0, 10× Denhardt's solution, 100 μg/ml thermally denatured salmon sperm DNA at 42° C.

In another aspect, the invention features a vector comprising a nucleic acid encoding a protein molecule shown in SEQ ID NO. 1. It also features a vector comprising a nucleic acid molecule encoding a protein molecule, the function of which is to protect cells against degeneration and/or cell death, wherein the amino acid sequence of the protein molecule comprises the sequence shown in SEQ ID NO. 1 or a functional variant thereof. In preferred embodiments, a virus, a bacteriophage, or a plasmid comprises the described nucleic acid. In particular, a plasmid adapted for expression in a bacterial cell comprises said nucleic acid molecule, e.g. a nucleic acid molecule encoding a protein molecule shown in SEQ ID NO. 1, and the regulatory elements necessary for expression of said molecule in the bacterial cell. In a further aspect, the invention features a plasmid adapted for expression in a yeast cell which comprises said nucleic acid molecule, e.g. a nucleic acid molecule encoding a protein molecule shown in SEQ ID NO. 1, and the regulatory elements necessary for expression of said molecule in the yeast cell. In another aspect, the invention features a plasmid adapted for expression in a mammalian cell which comprises a nucleic acid molecule, e.g. a nucleic acid molecule encoding a protein molecule shown in SEQ ID NO. 1, and the regulatory elements necessary for expression of said molecule in the mammalian cell.

In a further aspect, the invention features a cell comprising a nucleic acid molecule encoding a protein molecule shown in SEQ ID NO. 1. The invention also features cells comprising a nucleic acid molecule encoding a protein molecule whose function is to protect cells against degeneration and/or cell death and whose amino acid sequence comprises the sequence shown in SEQ ID NO. 1 or a functional variant thereof. It also features cells comprising a DNA molecule capable of hybridizing with the complement of the cDNA described in SEQ ID NO. 2 under stringent conditions. In preferred embodiments, said cell is a bacterial cell, a yeast cell, a mammalian cell, or a cell of an insect. In particular, the invention features a bacterial cell comprising a plasmid adapted for expression in a bacterial cell, said plasmid comprising a nucleic acid molecule which encodes a protein molecule shown in SEQ ID NO. 1, and the regulatory elements necessary for expression of said molecule in the bacterial cell. The invention also features a yeast cell comprising a plasmid adapted for expression in a yeast cell, said plasmid comprises a nucleic acid molecule encoding a protein molecule shown in SEQ ID NO. 1, and the regulatory elements necessary for expression of said molecule in the yeast cell. It further features a mammalian cell comprising a plasmid adapted for expression in a mammalian cell, said plasmid comprising a nucleic acid molecule which encodes a protein molecule shown in SEQ ID NO.1, and the regulatory elements necessary for expression of said molecule in the mammalian cell.

The invention further features an antibody specifically immunoreactive with an immunogen, wherein said immunogen is shown in SEQ ID NO. 1 or wherein said immunogen is a protein molecule, the function of which is to protect cells against degeneration and/or cell death, wherein the amino acid sequence of the protein molecule comprises the sequence shown in SEQ ID NO. 1 or a functional variant thereof. In another aspect, the invention aims at a method of detecting pathological cells in a subject which comprises immunocytochemically staining cells with the aforementioned antibody, wherein a low degree of staining in said cell compared to a reference cell representing a known health status indicates a pathological change of said cell. The invention is particularly suited to detect pathological structures in the brain of a subject—the detection method comprises immunocytochemically staining said pathological structures with said antibody. It is also especially suited to detect pathological cells of the muscular system, prostate, stomach, testis, ovary, adrenal glands, mammary glands, liver, spleen, lung, trachea or placenta.

In another aspect, the invention features a method of diagnosing or prognosing a disease, in particular a neurological disease, in a subject comprising:

determining a level, or an activity, or both said level and said activity, of at least one substance which is selected from the group consisting of

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(f) a translation product of a DNA molecule, wherein said DNA molecule is capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f),

and comparing said level, or said activity, or both said level and said activity, of at least one of said substances (a) to (h) to a reference value representing a known disease or health status, thereby diagnosing or prognosing a disease, in particular a neurological disease, in said subject.

In another aspect, the invention features a method of monitoring the progression of a disease, in particular a neurological disease, in a subject, comprising:

determining a level, or an activity, or both said level and said activity, of at least one substance which is selected from the group consisting of

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(f) a translation product of a DNA molecule, wherein said DNA molecule is capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f),

and comparing said level, or said activity, or both said level and said activity, of at least one of said substances (a) to (h) to a reference value representing a known disease or health status, thereby monitoring progression of a disease, in particular a neurological disease, in said subject.

In still a further aspect, the invention features a method of evaluating a treatment for a disease, in particular a neurological disease, in a subject, said method comprising:

determining a level, or an activity, or both said level and said activity, of at least one substance which is selected from the group consisting of

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said. DNA molecule is capable of hybridizing with the complement of the cDNA described in SEQ ID NO. 2 under stringent conditions,

(f) a translation product of a DNA molecule, wherein said DNA molecule is capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f),

and comparing said level, or said activity, or both said level and said activity, of at least one of said substances (a) to (h) to a reference value representing a known disease or health status, thereby evaluating a treatment for a disease, in particular a neurological disease, in said subject.

In a further aspect, the invention features a kit for diagnosis, or prognosis of a disease, said kit comprising:

(1) at least one reagent which is selected from the group consisting of reagents that selectively detect

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(f) a translation product of a DNA molecule, wherein said DNA molecule is capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f),

(2) instructions for diagnosing, or prognosing said disease by

(i) detecting a level, or an activity, or both said level and said activity, of at least one substance which is selected from the group consisting of (a) to (h) in a sample from said subject; and

(ii) diagnosing, or prognosing said disease, wherein

a varied level; or activity, or both said level and said activity, of at least one substance which is selected from the group consisting of (a) to (h) compared to a reference value representing a known health status;

or a level, or activity, or both said level and said activity, of at least one substance which is selected from the group consisting of (a) to (h) similar or equal to a reference value representing a known disease status indicates diagnosis, or prognosis of said disease.

In a further aspect, the kit may be used in monitoring success or failure of a therapeutic treatment of said subject. It can also be used in monitoring the progression of a disease.

Preferred embodiments of the above mentioned methods and kit of diagnosing or prognosing diseases, or monitoring the progression thereof, or evaluating a treatment thereof, are now disclosed in detail.

In a preferred embodiment, the function of said protein molecule or a functional variant thereof is to protect cells from degeneration and/or cell death.

In another preferred embodiment, said DNA molecule capable of hybridizing with the complement of the cDNA described in SEQ ID NO. 2 encodes a protein molecule, the function of which is to protect cells against cell degeneration and/or cell death.

In preferred embodiments, said subjects suffer from Alzheimer's disease and related neurofibrillary disorders, or degenerative states, e.g. neurodegenerative states, characterized by cell degeneration or cell death. Further examples of neurological diseases are Parkinson's disease, Huntington disease, amyotrophic lateral sclerosis and Pick's disease.

It is particularly preferred that said sample is a brain tissue or other body cells including cells of the muscular system, prostate, stomach, testis, ovary, adrenal glands, mammary glands, liver, spleen, lung, trachea, or placenta. The sample might also be cerebrospinal fluid or another body fluid.

According to the present invention, a reduction in the level, or activity, or both said level and said activity, of (i) a transcription product of a DNA molecule encoding a protein molecule, whose amino acid sequence comprises the sequence shown in SEQ ID NO.1 or a functional variant thereof or (ii) a protein molecule whose amino acid sequence comprises the sequence-shown in SEQ ID NO. 1 or a functional variant thereof, in a sample from said subject relative to a reference value representing a known health status indicates the presence of a pathological status in said subject. In particular, a reduction in the level, or activity, or both said level and said activity of SELADIN-1 or SELADIN-1 transcripts in said subject's brain regions affected heavily by neurodegeneration relative to a reference value representing a known health status indicates a diagnosis or prognosis of Alzheimer's disease. Predominantly neurons within the inferior temporal lobe, the entorhinal cortex, the hippocampus and the amygdala degenerate in Alzheimer's disease.

It might be preferred that said subject has previously been determined to have one or more factors indicating that such subject is afflicted with a disease under study, in particular a neurological disease.

In preferred embodiments, said subject can be a human, an experimental animal, e.g. a rat or a mouse, a domestic animal, or a non-human primate, e.g. a monkey. The experimental animal can be an animal model for a disorder, e.g. a transgenic mouse with an Alzheimer's-type neuropathology.

In preferred embodiments, at least one of said substances is detected using an immunoassay, an enzyme activity assay and/or a binding assay.

In preferred embodiments, measurement of the level of transcription products of the SELADIN-1 gene, or a functional variant thereof, is performed in body cells using Northern blots with probes specific for the SELADIN-1 gene or said variant. Quantitative PCR with primer combinations to amplify SELADIN-1 gene-specific sequences from cDNA obtained by reverse transcription of RNA extracted from body cells of a subject can also be applied. These techniques are known to those of ordinary skill in the art (see e.g. Watson et al., Rekombinierte DNA, 2nd edition, Spektrum Akademischer Verlag GmbH, Heidelberg, 1993; Watson et al., Recombinant DNA, 2nd ed. W.H. Freeman and Company, 1992).

In preferred embodiments, said level or activity of the protein molecule shown in SEQ ID NO. 1, or a functional variant or fragment thereof, is detected using an immunoassay. These assays can measure the amount of binding between said protein molecule and an anti-protein antibody, e.g. an anti-SELADIN-1 antibody, by the use of enzymatic, chromodynamic, radioactive, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow et al., Antibodies, A Laboratory Manual, Cold Spring Harbor Laboratory Press, Cold Spring Harbor, N.Y.).

The antibody or ligand to be used should preferably specifically detect SELADIN-1 or a functional variant or fragment thereof. It is preferred that it does not substantially interact with any other protein present in said sample.

Monoclonal antibodies capable of recognizing a protein molecule of SEQ ID NO. 1 or a functional variant or fragment thereof can be prepared using methods known in the art (see e.g. Kohler and Milstein, Nature 256, 495-497 1975; Kozbor et al., Immunol. Today 4, 72, 1983; Cole et al., Monoclonal antibodies and cancer therapy, Alan R. Liss, Inc., pp 77-96, 1985; Marks et al., J. Biol. Chem., 16007-16010, 1992; the contents of which are incorporated herein by reference). Such monoclonal antibodies or fragments thereof can also be produced by alternative methods known to those of skill in the art of recombinant DNA technology (see e.g. Sastry et al, PNAS 86: 5728, 1989; Watson et al., Rekombinierte DNA, 2nd ed., Spektrum Akademischer Verlag GmbH, 1993; Watson et al, Recombinant DNA, 2nd ed., W. H. Freeman and Company, 1992; the contents of which are incorporated herein by reference). Monoclonal antibodies useful in the methods of the invention are directed to an epitope of SELADIN-1 or a functional variant or fragment thereof, such that the complex formed between the antibody and SELADIN-1, or between the antibody and said functional variant or fragment, can be recognized in detection assays. The term “antibodies” encompasses all forms of antibodies known in the art, such as polyclonal, monoclonal, chimeric, recombinatorial, single chain antibodies as well as fragments thereof which specifically bind to SELADIN-1, or to a functional variant or fragment thereof.

Antibodies or ligands might also be used in detecting specifically molecules mentioned in the above described methods and kit under g) and h) above.

If luminescent labels are used in any detection assay, it is preferred to use a confocal optical set-up.

In further preferred embodiments, said reference value is that of a level, or an activity, or both said level and said activity, of at least one substance which is selected from the group consisting of (a) to (h) described above in a sample from a subject not suffering of the disease under study, in particular a neurological disease such as Alzheimer's disease. The healthy subject can be of the same weight, age, and gender as the subject who is being diagnosed or prognosed for said disease. In some cases, it might be preferred to use a reference value from the subject which is diagnosed.

In a preferred embodiment, the level, or the activity, or both said level and said activity, of at least one of said substances (a) to (h) described above in a sample is determined at least twice, e.g. at two points which are weeks or months apart. The levels or activities at these two time points are compared in order to monitor the progression of said disease. It might be preferred to take a series of samples over a period of time. In further preferred embodiments, said subject receives a treatment prior to one or more of said sample gatherings.

In another aspect, the invention features a method of treating or preventing a disease, in particular a neurological disease, in a subject comprising administering to said subject in a therapeutically effective amount an agent or agents which affect a level, or an activity, or both said level and said activity, of at least one substance which is selected from the group consisting of

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(f) a translation product of a DNA molecule, wherein said DNA molecule is capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f).

In a preferred embodiment, the function of said protein molecule or a functional variant thereof is to protect cells from degeneration and/or cell death.

In another preferred embodiment, said DNA molecule capable of hybridizing with the complement of the cDNA described in SEQ ID NO. 2 encodes a protein molecule, the function of which is to protect cells against cell degeneration and/or cell death.

In preferred embodiments, said subjects suffer from Alzheimer's disease and related neurofibrillary disorders, or degenerative states, such as neurodegenerative states, characterized by cell degeneration or cell death. Further examples of neurological diseases are Parkinson's disease, Huntington disease, amyotrophic lateral sclerosis and Pick's disease.

In preferred embodiments, the method comprises the application of per se known methods of gene therapy nucleic acid technology to administer said agent or said agents.

In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc. Chem. Res. 26, 274-278, 1993; Mulligan, Science 260, 926-931, 1993; the contents of which are incorporated herein by reference) and include direct gene-transfer techniques such as mechanical microinjection of DNA into a cell as well as indirect techniques employing biological vectors (like recombinant viruses, especially retro viruses) or model liposomes, or techniques based on transfection with DNA coprecipitation with polycations, cell membrane perturbation by chemical (solvents, detergents, polymers, enzymes) or physical means (mechanic, osmotic, thermic, electric shocks). The postnatal gene transfer into the central nervous system has been described in detail (see e.g. Wolff, Current Opinion in Neurobiology, 3, 743-748, 1993; the contents of which are incorporated herein by reference).

In preferred embodiments, the method comprises grafting donor cells into the central nervous system, preferably the brain, of said subject, said subject or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection, liposomal mediated transfection, etc.

In preferred embodiments, said agent is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

In preferred embodiments, the therapeutic nucleic acid or protein reduces or prevents the degeneration of cells, in particular neurons and slows brain amyloid formation.

In another aspect, the invention features an agent which affects an activity, or level, or both said activity or level, of at least one substance which is selected from the group consisting of:

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(f) a translation product of a DNA molecule capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f).

Preferably, the function of said protein molecule or a variant thereof is to protect cells from degeneration and/or cell death. Preferably, said DNA molecule capable of hybridizing with the complement of the cDNA described in SEQ ID NO. 2 encodes a protein, whose function is to protect cells from degeneration and/or cell death.

In another aspect, the invention features a medicament comprising such an agent.

In still another aspect, the invention features an agent for treating or preventing a disease, in particular a neurological disease, which agent affects an activity, or level, or both said activity or level, of at least one substance which is selected from the group consisting of

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(f) a translation product of a DNA molecule, wherein said DNA molecule is capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f).

In preferred embodiments, said diseases are degenerative states characterized by cell degeneration or cell death or Alzheimer's disease and related neurofibrillary disorders. Further examples of neurological diseases are Parkinson's disease, Huntington disease, Amyotrophic lateral sclerosis, Pick's disease.

Preferably, the function of said protein molecule or a variant thereof is to protect cells from degeneration and/or cell death. Preferably, said DNA molecule capable of hybridizing with the complement of the cDNA described in SEQ ID NO. 2 encodes a protein, whose function is to protect cells from degeneration and/or cell death.

In a further aspect, the invention features the use of an agent, for preparation of a medicament for treating or preventing a neurological disease, which agent affects an activity, or level, or both said activity or level, of at least one substance which is selected from the group consisting of

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(g) a translation product of a DNA molecule, wherein said DNA molecule is capable of hybridizing with the complement of the cDNA described in SEQ ID NO. 2 under stringent conditions,

(h) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f).

Preferably, the function of said protein molecule or a variant thereof is to protect cells from degeneration and/or cell death. Preferably, said DNA molecule capable of hybridizing with the complement of the cDNA described in SEQ ID NO.2 encodes a protein molecule, whose function is to protect cells against degeneration and/or cell death.

In preferred embodiments, said diseases are Alzheimer's disease and related neurofibrillary disorders, or degenerative states, in particular neurodegenerative states, characterized by cell degeneration or cell death. Further examples of neurological diseases are Parkinson's disease, Huntington disease, Amyotrophic lateral sclerosis, Pick's disease.

In a further aspect, the invention features a method for identifying an agent that affects an activity, or level, or both said activity or level, of at least one substance which is selected from the group consisting of

(a) a DNA molecule encoding a protein molecule, wherein the amino acid sequence of the protein molecule comprises the sequence shown in SEQ ID NO.1 or a functional variant thereof,

(b) a transcription product of a DNA molecule encoding a protein molecule, wherein the amino acid sequence of the protein molecule comprises the sequence shown in SEQ ID NO.1 or a functional variant thereof,

(c) a protein molecule, wherein the amino acid sequence of the protein molecule comprises the sequence shown in SEQ ID NO.1 or a functional variant thereof,

(d) a DNA molecule capable of hybridizing with the complement of the cDNA A described in SEQ ID NO. 2 under stringent conditions,

(e) a transcription product of a DNA molecule, wherein said DNA molecule is capable of hybridizing with the complement of the cDNA described in SEQ ID NO. 2 under stringent conditions,

(f) a translation product of a DNA molecule, wherein said DNA molecule is capable of hybridizing with the complement of the cDNA described in SEQ ID NO. 2 under stringent conditions,

(g) a molecule affecting a level, or an activity, or both said level and said activity, of at least one substance which is selected from the group consisting of (a) to (f),

(h) a molecule which is affected in its level, or its activity, or both its level and activity, by at least one substance which is selected from the group consisting of (a) to (f),

comprising the steps of:

(i) providing a sample containing at least one substance which is selected from the group consisting of (a) to (f),

(ii) contacting said sample with at least one agent,

(iii) comparing an activity, or level, or both said activity and level, of at least one of said substances before and after contacting.

Preferably, the function of said protein molecule or a variant thereof is to protect cells from degeneration and/or cell death. Preferably, said DNA molecule capable of hybridizing with the complement of the cDNA described in SEQ ID NO. 2 encodes a protein molecule, whose function is to protect cells against degeneration and/or cell death.

Other features and advantages of the invention will be apparent from the following detailed description of the figures, the examples and the claims.

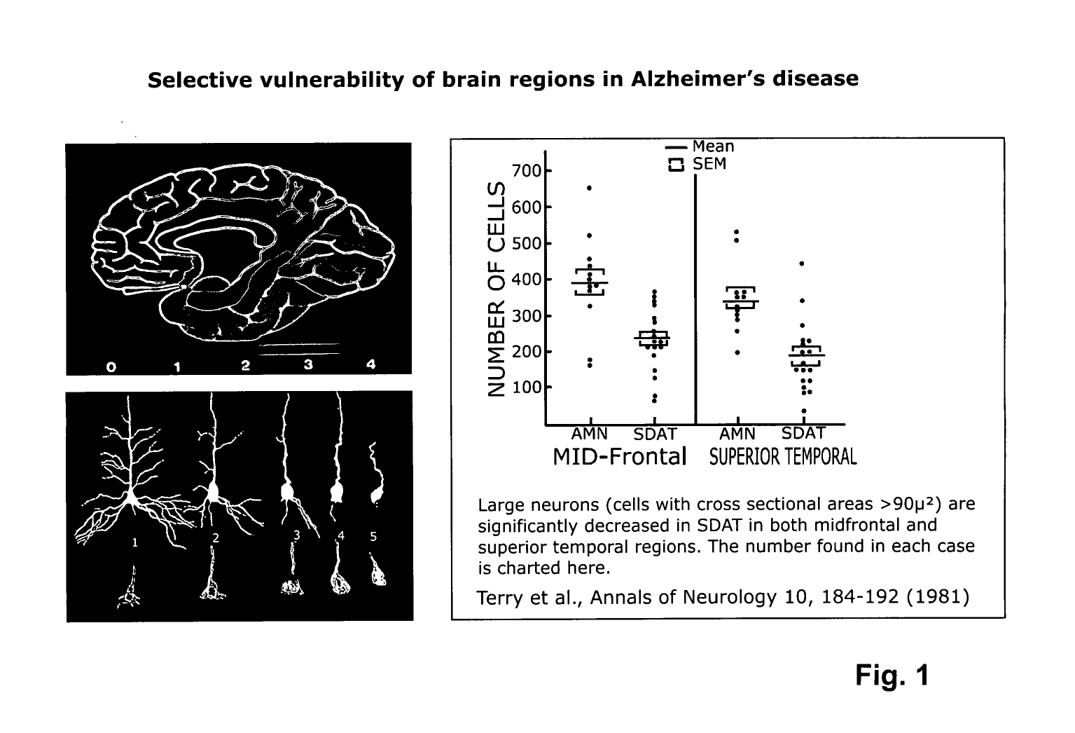

FIG. 1 depicts the selective vulnerability of brain regions in Alzheimer's disease. Predominantly neurons within the inferior temporal lobe, the entorhinal cortex, the hippocampus and the amygdala degenerate in Alzheimer's disease (Terry et al., Annals of Neurology, 10, 184-192, 1981). These brain regions are predominantly involved in the processing of learning and memory functions. In contrast, neurons within the frontal cortex, the occipital cortex and the cerebellum are largely intact and preserved from the neurodegenerative process in Alzheimer's disease.

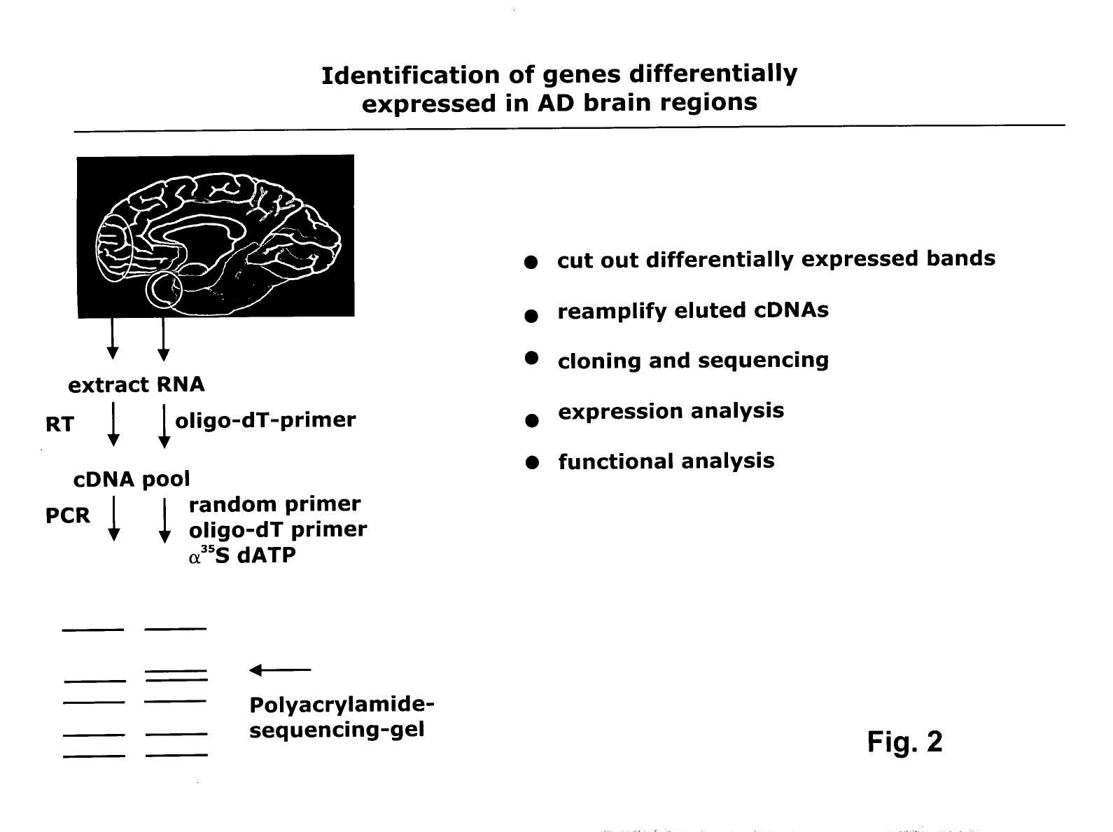

FIG. 2 discloses the identification of genes differentially expressed in brain regions from Alzheimer's disease patients. Brain areas with massive neuronal cell loss as well as areas with largely preserved neurons were identified and RNA extracted. Synthesis of cDNA was performed using an oligo-dT primer followed by PCR using the oligo-dT primer in combination with random primers and (α³⁵S)-dATP. Reactions were separated on DNA sequencing gels, DNA bands visualized by autoradiography and bands lighting up in different intensities were cut out. DNA fragments were reamplified by PCR, cloned in E. coli and sequences determined. Expression and functional analyses were performed.

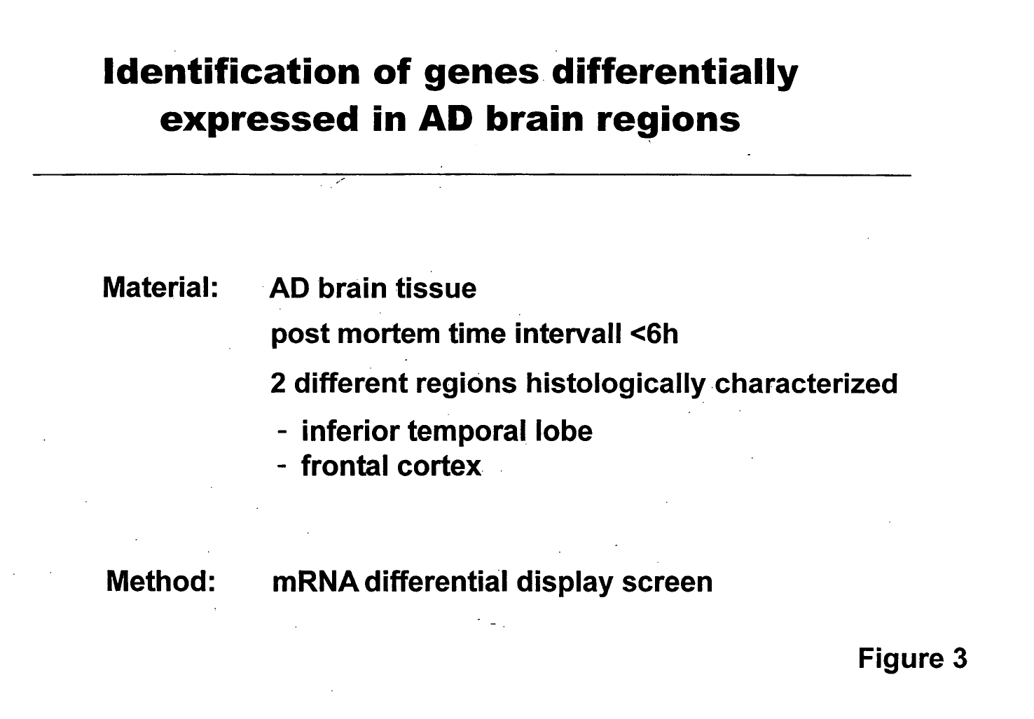

FIG. 3 depicts the specifications of Alzheimer's disease brain tissue as it was used in the examples. Brain tissues from Alzheimer's disease patients and control subjects were removed within 6 hours of death, and immediately frozen on dry ice. For RNA extraction tissue sections from the inferior temporal lobe and frontal cortex were chosen.

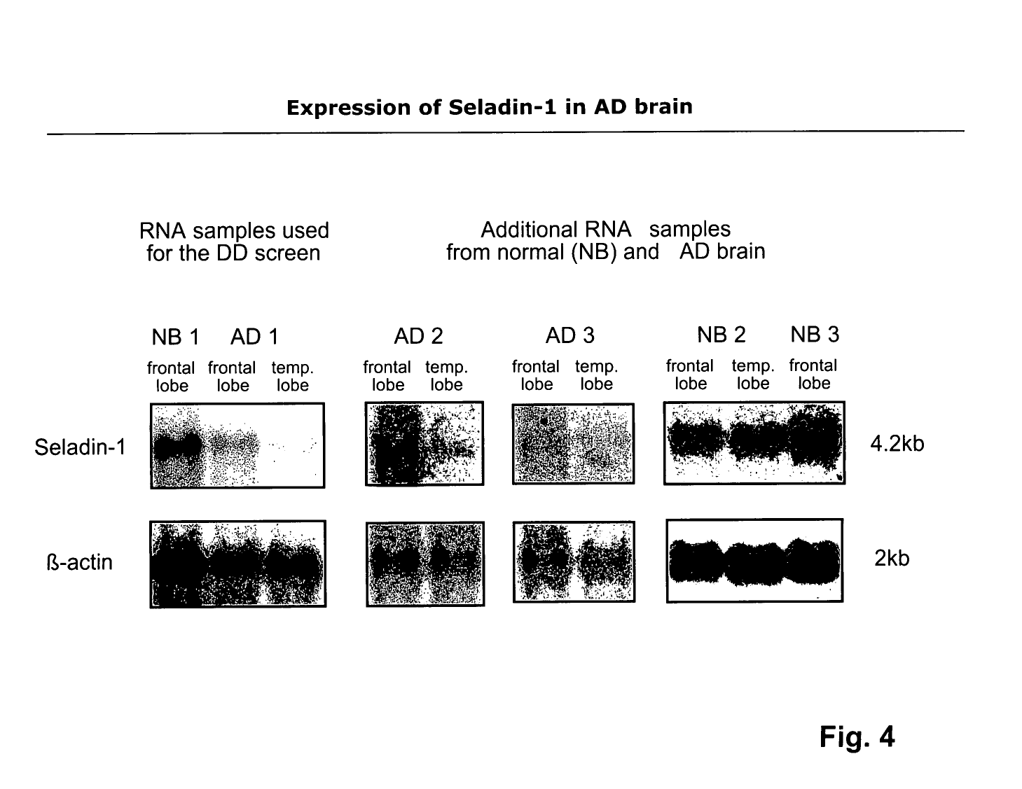

FIG. 4 discloses the quantification of SELADIN-1 transcripts in brain tissue from Alzheimer's disease and control subjects by Northern blot analyses. Transcript levels were significantly lower in brain regions with severe neurodegeneration, i.e. temporal lobe in Alzheimer's disease (AD1-3) but not in normal brain (NB1-3), as compared to protected brain regions, i.e. frontal lobe. This decrease was specific as indicated by unchanged β-actin transcript levels used to control for equal loading of RNA.

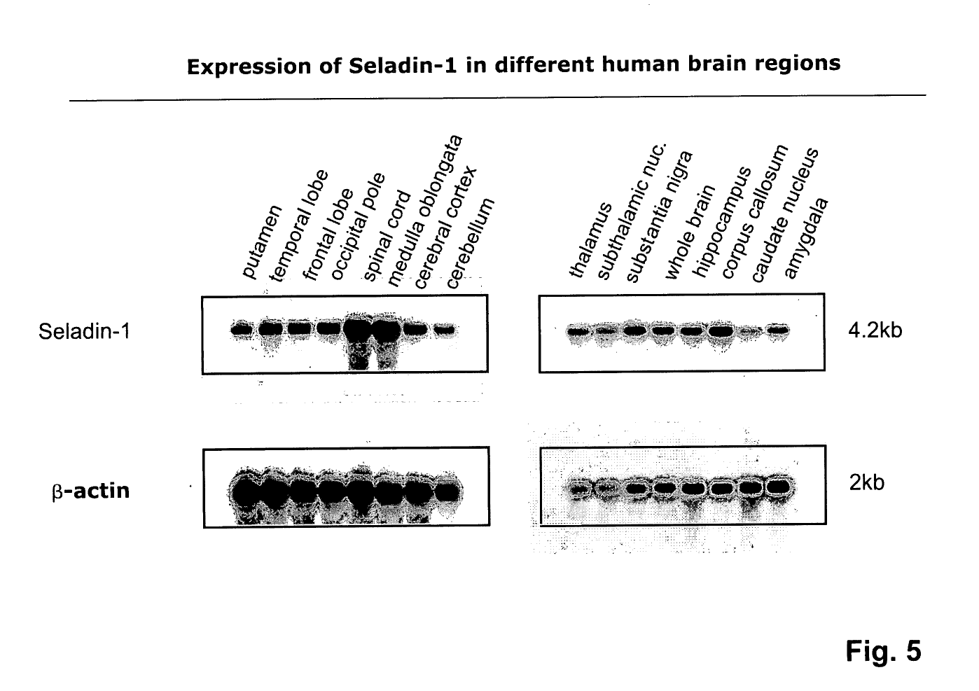

FIG. 5 depicts the transcription levels of the SELADIN-1 gene in different human brain regions. The SELADIN-1 gene was found to be expressed throughout the human brain. In particular transcription levels are high in all cortical areas, the hippocampus, the amygdala, the spinal cord, and the medulla. Note the unchanged levels in temporal lobe versus frontal lobe in this brain derived from a cognitively normal control subject without any signs of Alzheimer's disease. The analysis of β-actin transcripts was used as loading control.

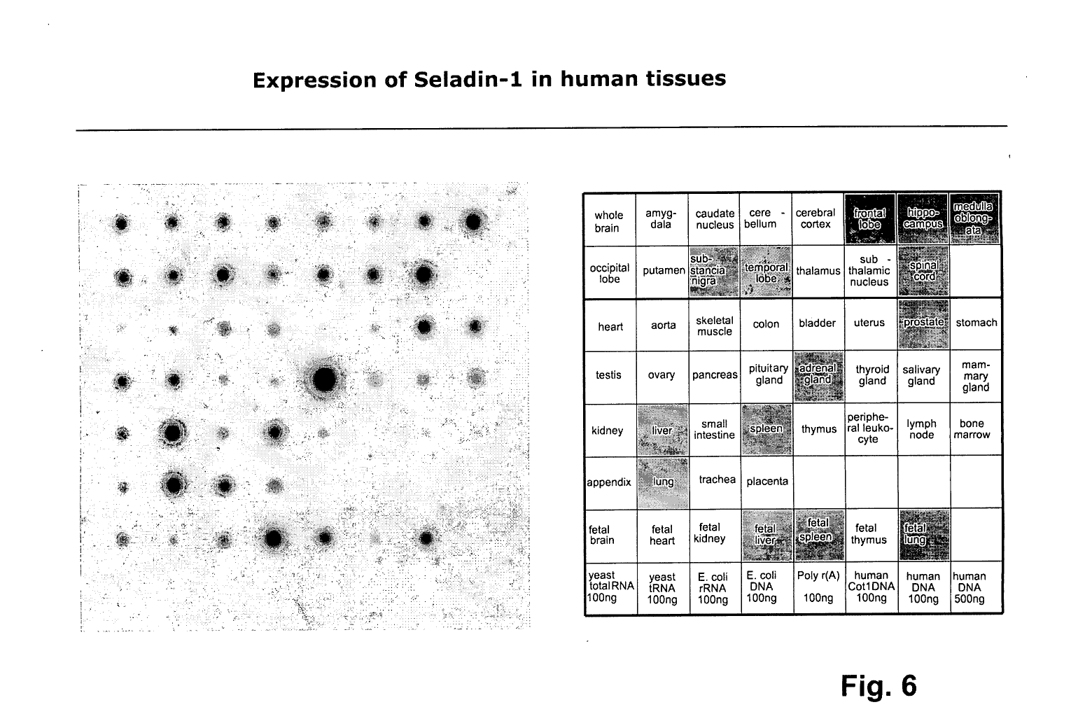

FIG. 6 depicts the distribution of SELADIN-1 transcripts in human tissues. Comparable samples of RNA were spotted on nitrocellulose filters and SELADIN-1 transcripts were quantified by hybridization using a labeled SELADIN-1 gene specific probe. Significant levels of SELADIN-1 gene transcripts were found in all brain regions tested. Transcripts were also detected in other tissues, however, strong variations in signal intensity indicated a tissue specific regulation of SELADIN-1 expression.

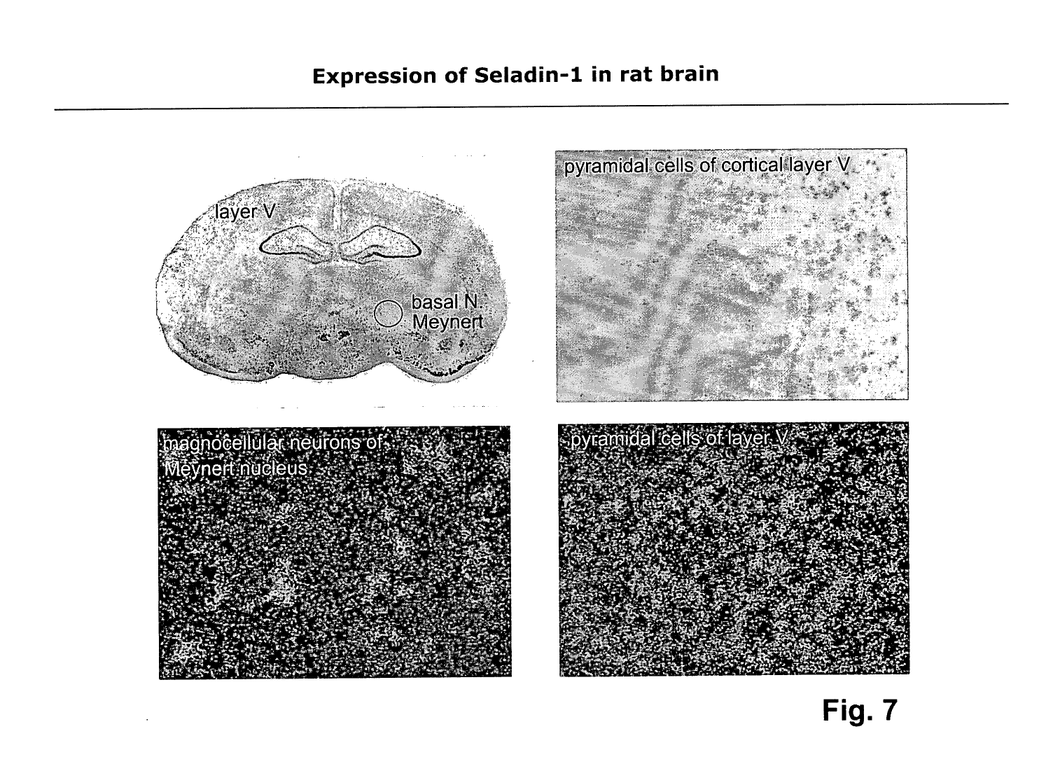

FIG. 7 depicts the expression of the SELADIN-1 gene in rat brain cortex, hippocampus and basal nucleus analyzed by in situ hybridization. This staining pattern along with the higher magnifications indicate that SELADIN-1 is predominantly expressed in neurons. No significant hybridization signals were observed with glial cells.

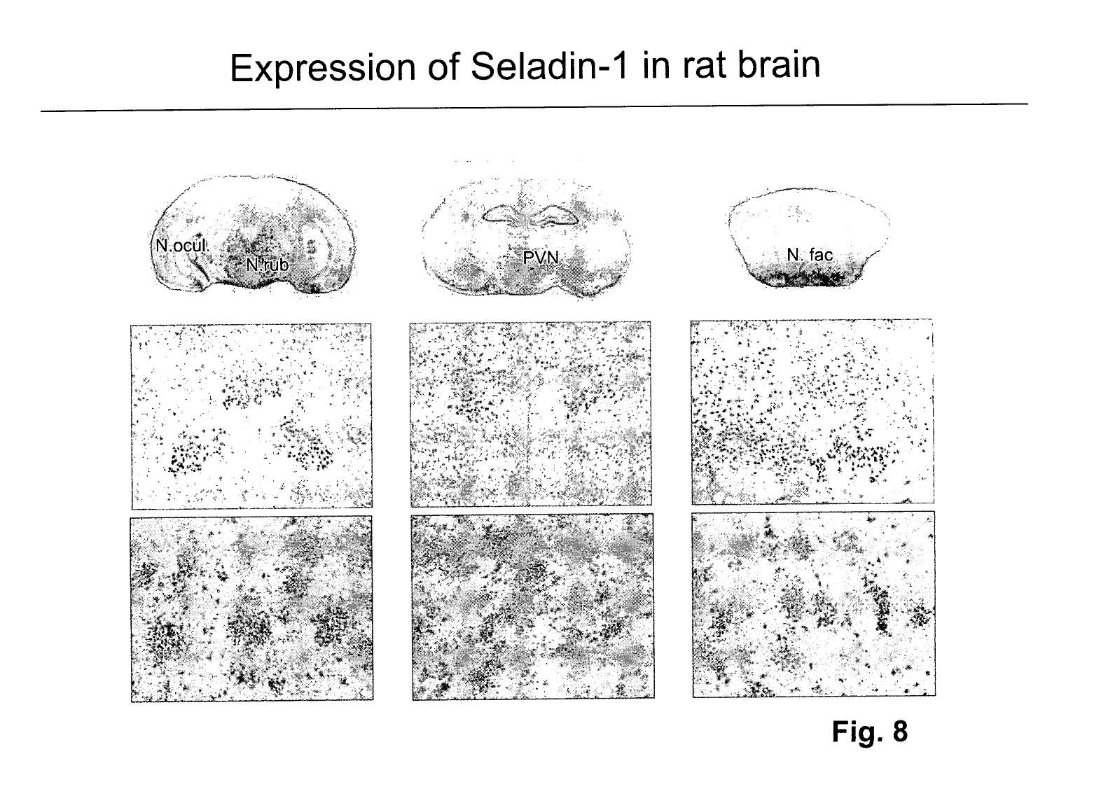

FIG. 8 depicts the expression of SELADIN-1 in rat brain nuclei. Strong SELADIN-1 expression was found in the oculomotor, paraventricular, red and facial nuclei. Higer magnifications indicate predominant hybridization with neurons. No significant hybridization signals were observed with glial cells.

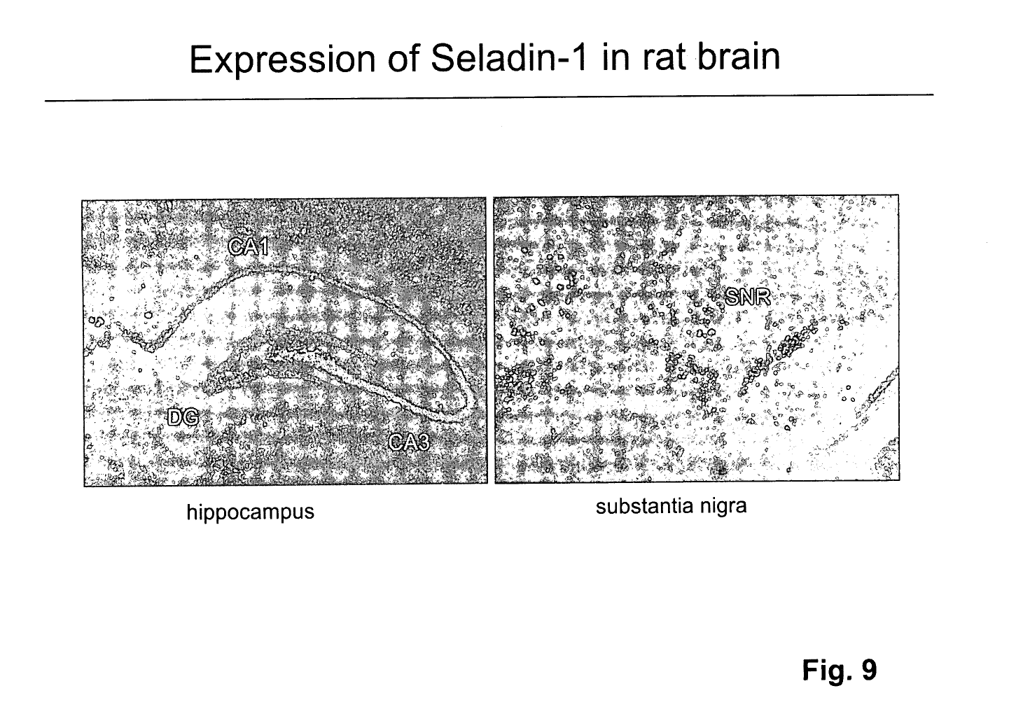

FIG. 9 depicts the expression of SELADIN-1 in rat brain hippocampus and substantia nigra. in situ hybridization with SELADIN-1 transcripts was detected by photoemulsion autoradiography, confirming the neuron specific expression of this gene.

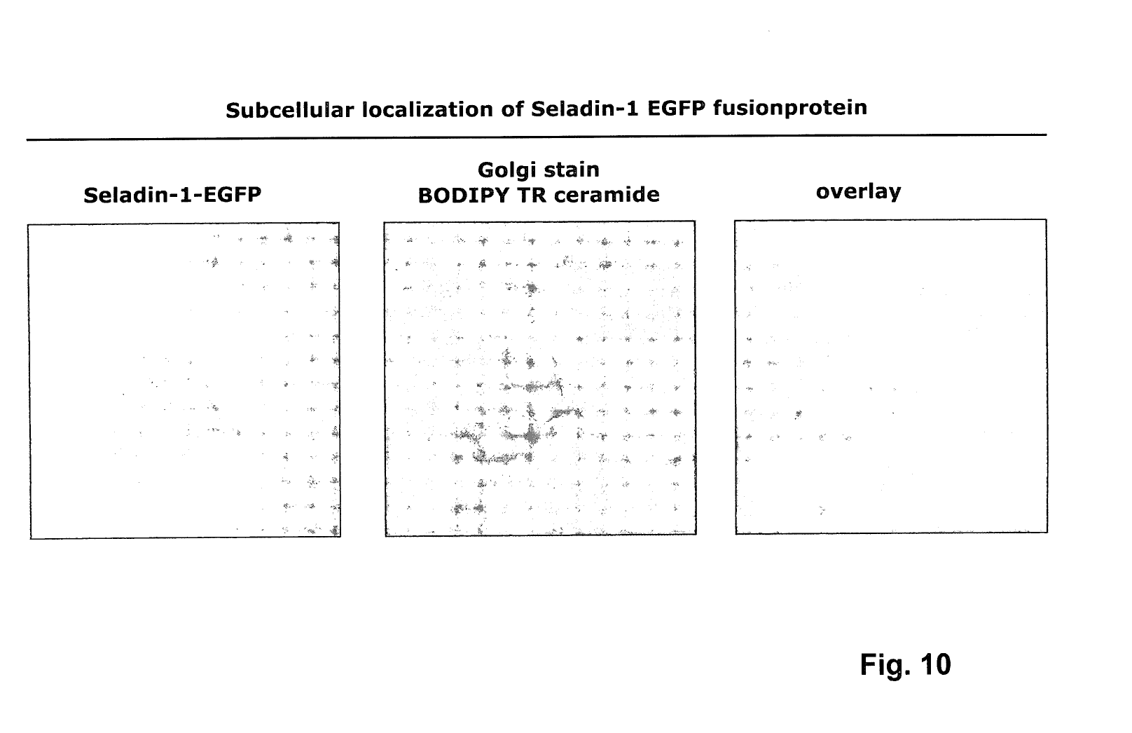

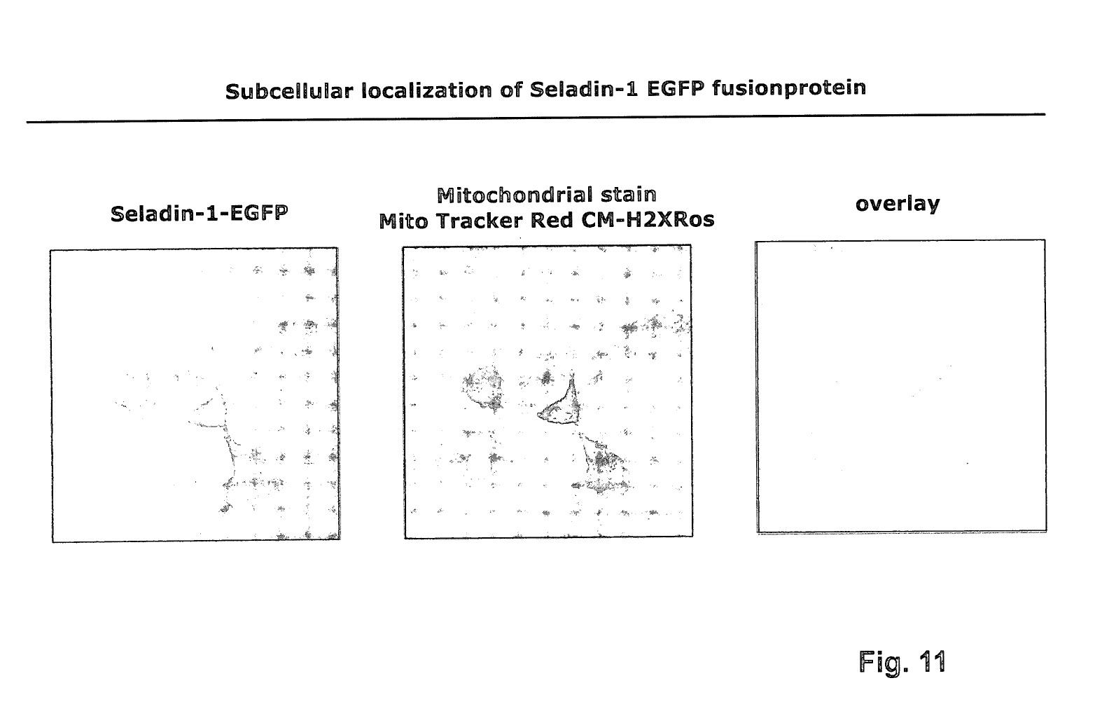

FIG. 10 discloses the subcellular localization of a SELADIN-1-EGFP (enhanced green fluorescent protein) fusion in transfected cos cells. The confocal micrographs show the co-localization of the SELADIN-1-EGFP fusion with the golgi specific stain BODIPY TR ceramide indicating localization of SELADIN-1 in the Golgi apparatus and the endoplasmic reticulum.

FIG. 11 discloses that the SELADIN-1-EGFP fusion does not localize to mitochondria in transfected cos cells in spite of a putative mitochondrial targeting sequence close to the N-terminus of the SELADIN-1 protein. The confocal micrographs show the different staining patterns caused by the SELADIN-1-EGFP fusion and the specific mitochondrial stain Mito Tracker Red CM-H₂XRos.

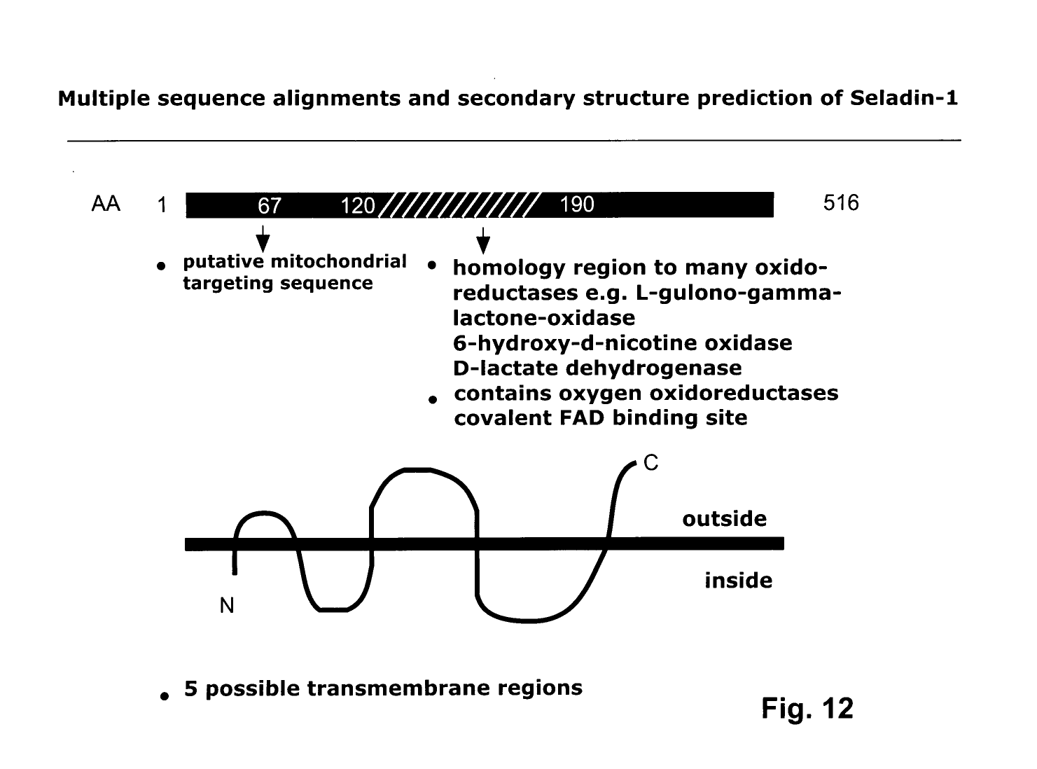

FIG. 12 discloses structural features of the SELADIN-1 protein based on multiple sequence alignments and secondary structure predictions. Near the N-terminus the SELADIN-1 protein contains a putative mitochondrial localization signal that appears to be inactive in transfected cos cells or when used in EGFP fusions. The central region of the protein contains a sequence that is homologous to a family of oxidoreductases and that contains a FAD site for covalent binding. The protein is predicted to contain five transmembrane regions. The expression in neurons, the co-localization in the Golgi apparatus and the endoplasmic reticulum of the SELADIN-1 protein, the amyloid precursor protein (APP) and the presenilins PS1 and PS2 and furthermore the transmembrane character suggest a functional relationship between these proteins. Mutations in both APP and presenilins were shown to cause an increase in the production of β-amyloid. In a similar way the SELADIN-1 protein might be involved in common biological pathways influencing the processing of the amyloid precursor protein and the generation of Aβ. Using the SELADIN-1 protein as a probe, interaction partners can be identified which might represent new AD drug targets.

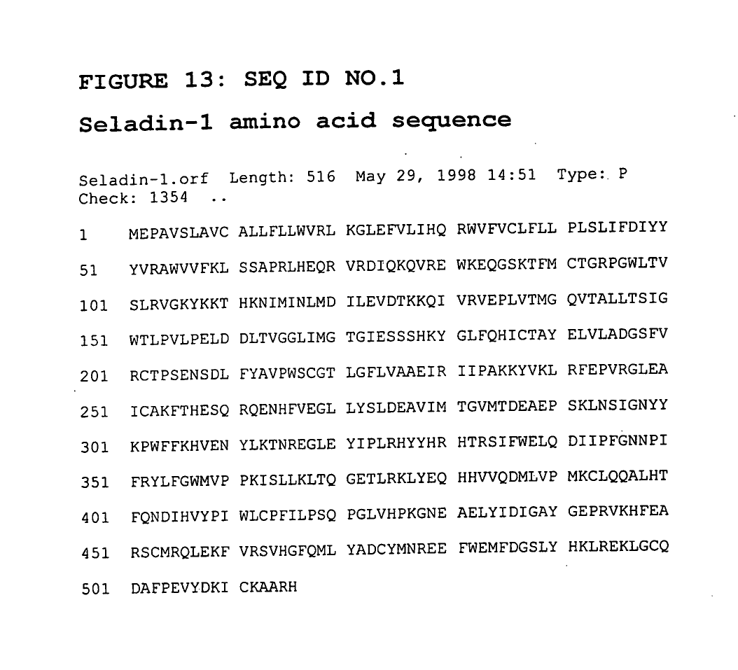

FIG. 13 discloses the protein sequence of SELADIN-1 (SEQ ID NO. 1). The full length protein consists of 516 amino acid residues. The sequence is given in the one letter amino acid code.

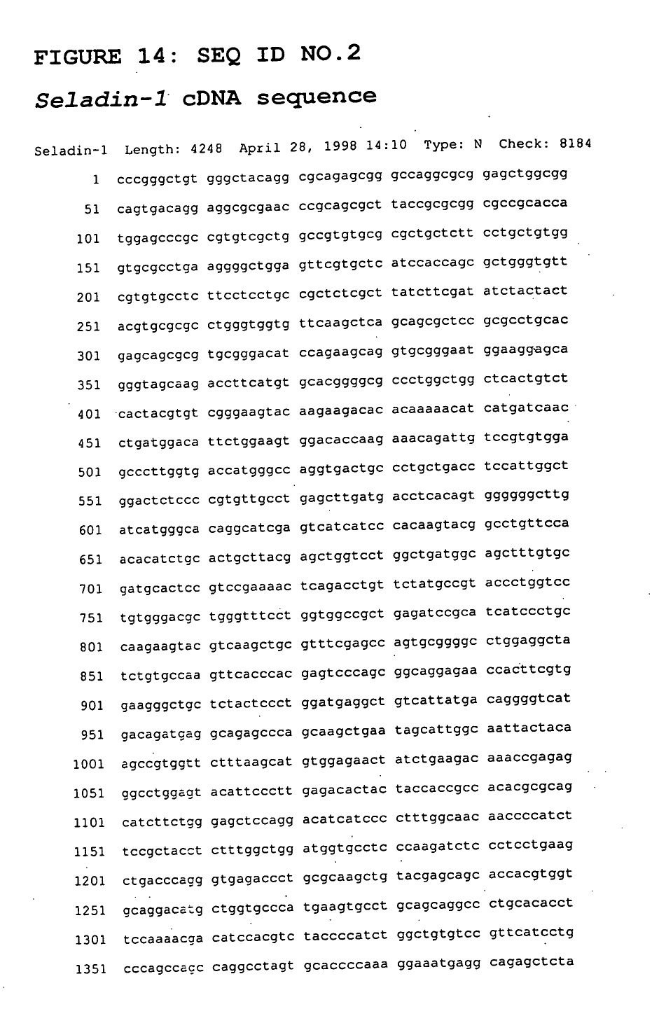

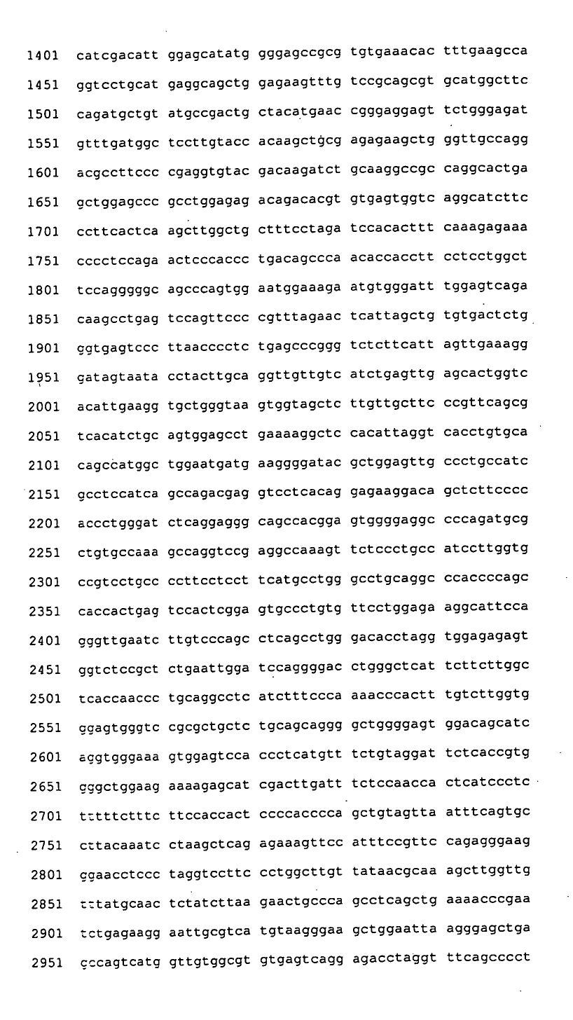

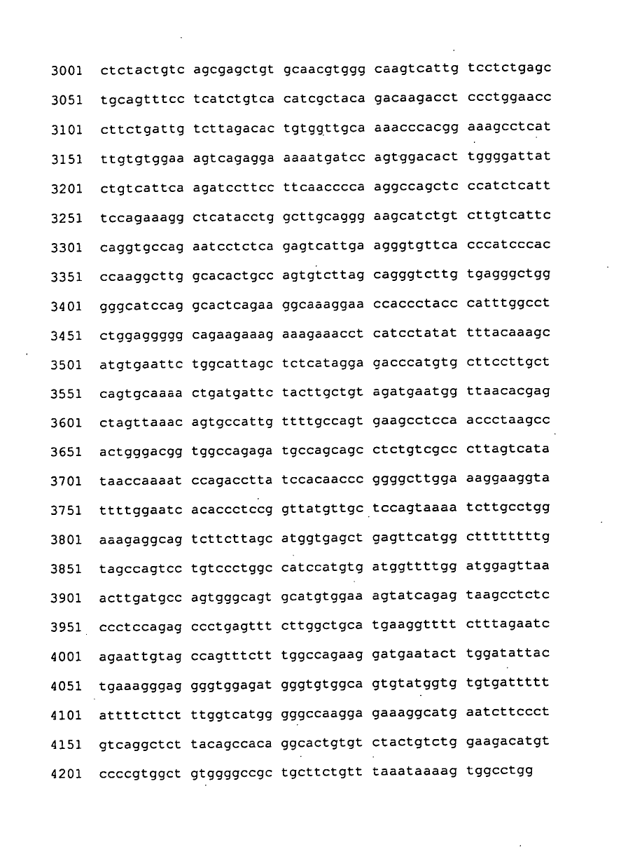

FIG. 14 discloses the nucleotide sequence of the cloned SELADIN-1 cDNA (SEQ ID NO. 2) comprising 4248 nucleotides. The coding sequence for the SELADIN-1 protein starts at nucleotide position 100 and stops at position 1648.

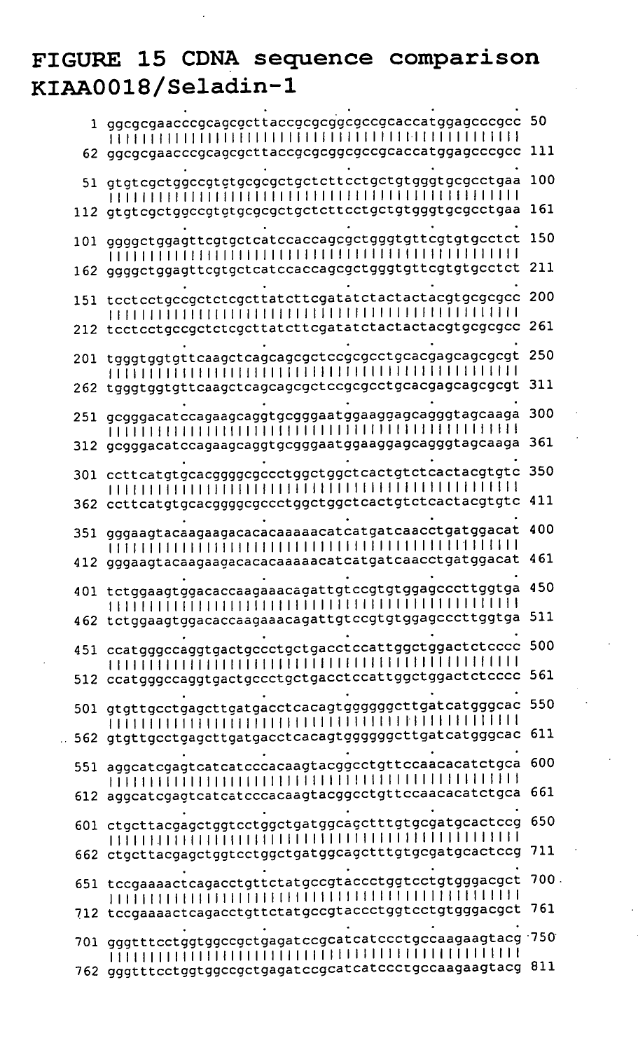

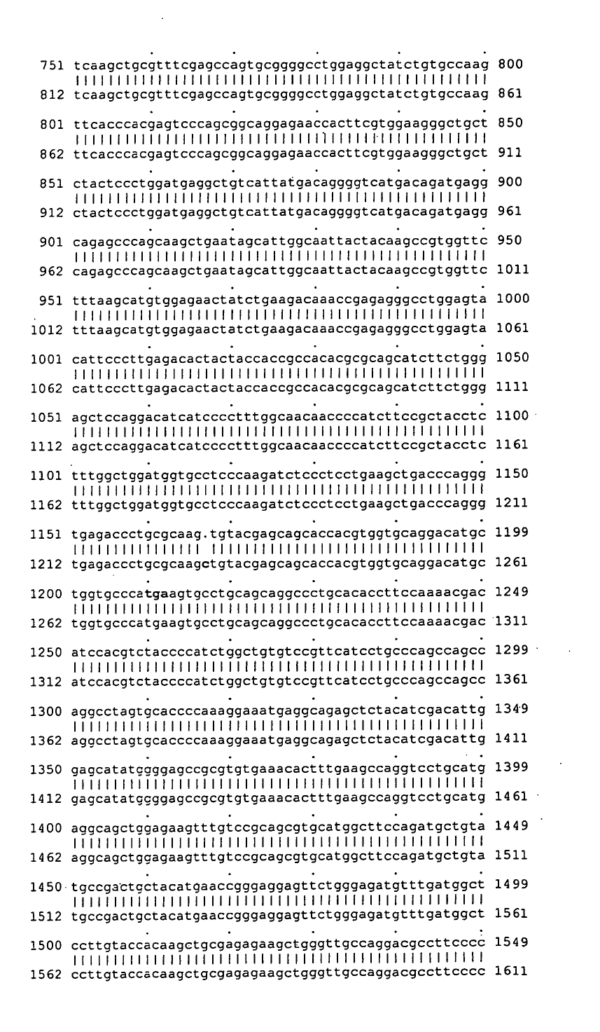

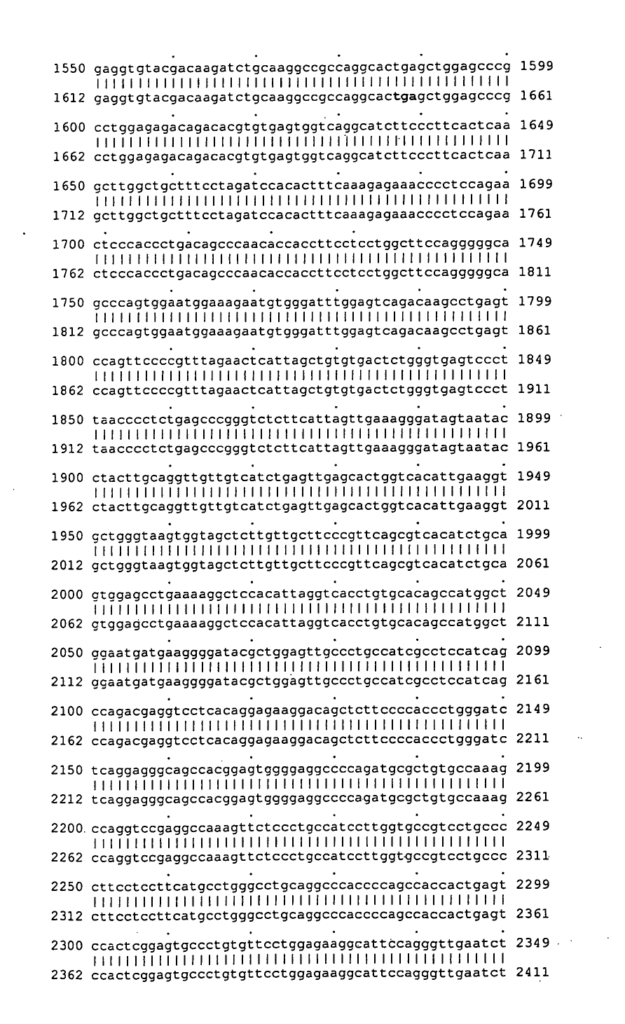

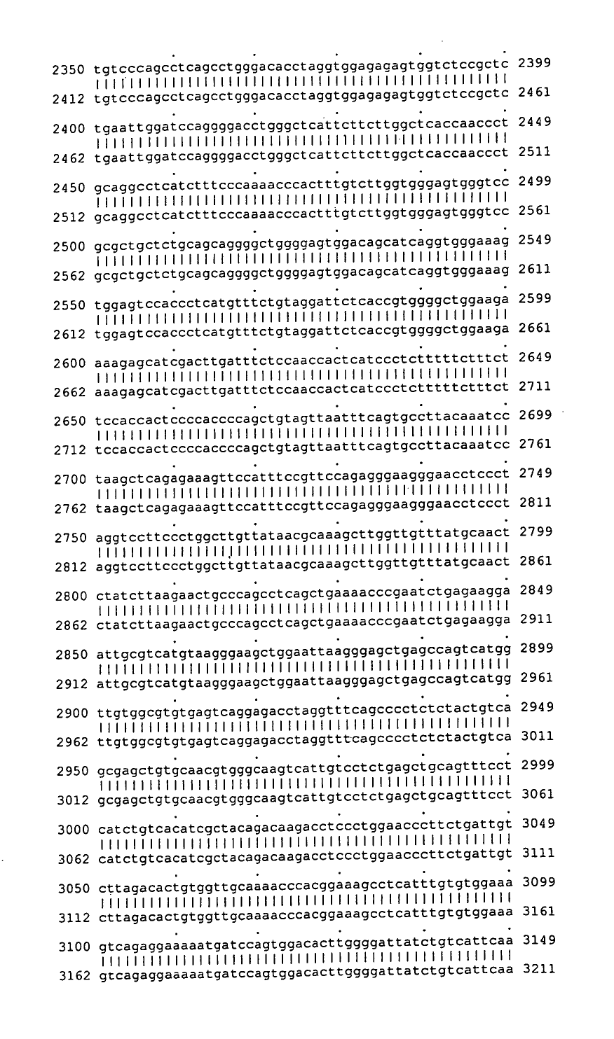

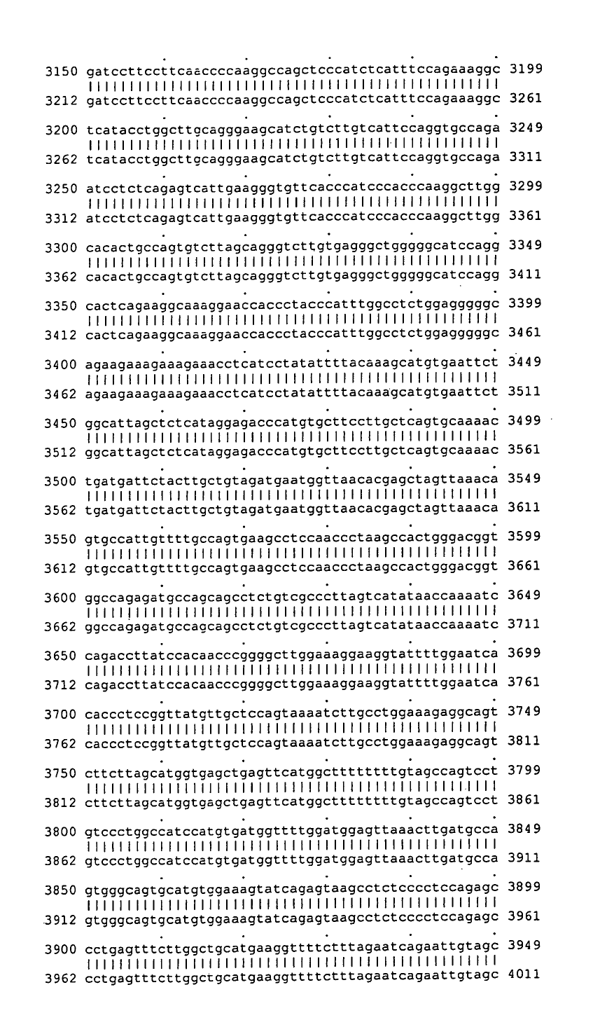

FIG. 15 discloses the comparison of nucleotide sequences of the cloned SELADIN-1 cDNA comprising 4248 nucleotides and the KIAA0018 cDNA comprising 4186 nucleotides. A significant difference exists at position 1228 of the SELADIN-1 sequence where a C nucleotide (C/G base pair) is missing in the KIAA0018 sequence. This results in a frame shift in the open reading frame in the KIAA0018 sequence relative to the SELADIN-1 sequence. The consequence is that the translation product of the KIAA0018 gene is 390 amino acids in length compared to 516 amino acid residues of the SELADIN-1 translation product. In addition to the difference in length, the frame shift causes a difference between the C-terminal 14 amino acids of the KIAA0018 protein and the corresponding sequence area of the SELADIN-1 polypeptide (pos. 377-390). The coding sequence for the SELADIN-1 protein starts at nucleotide position 100 and stops at position 1648.

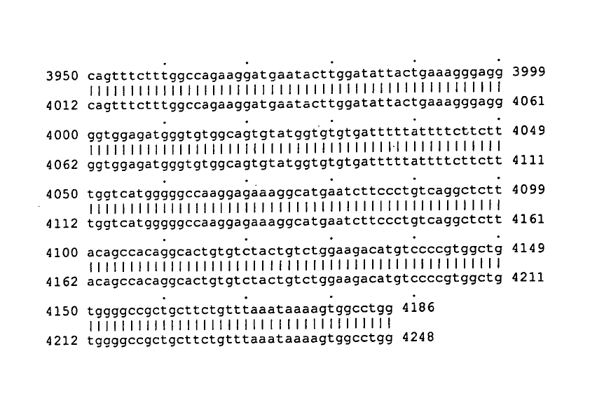

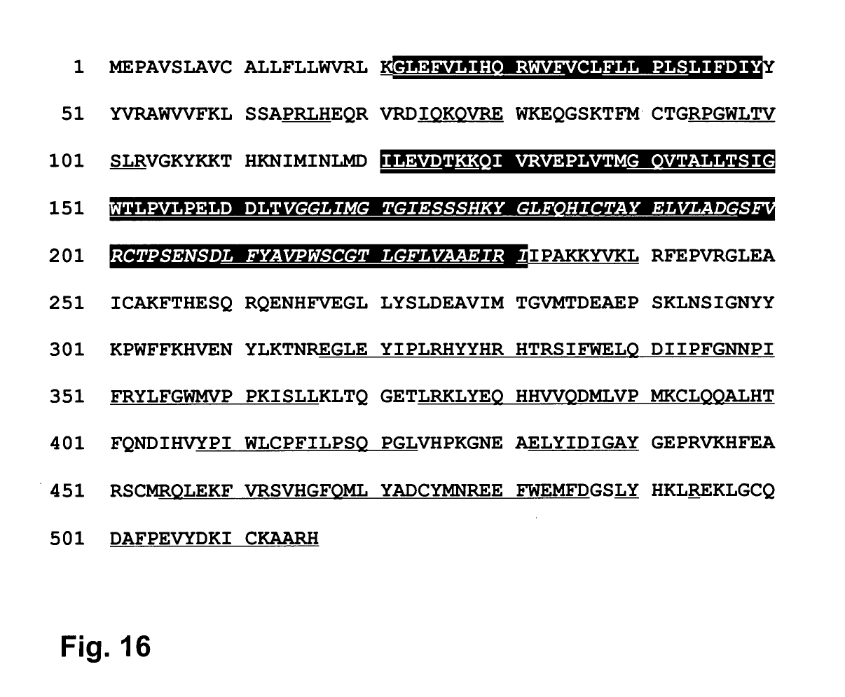

FIG. 16 shows the amino acid sequence of SELADIN-1. A differential display approach (von der Kammer, H. et al., Nucleic acid research, 27, 2211, 1999; von der Kammer, H. et al., J. Biol. Chem. 273, 14538, 1998) to identify genes that are differentially expressed in selectively vulnerable cell populations in the inferior temporal cortex with confirmed neurodegeneration and in the largely unaffected frontal or sensory-motor cortex of the same subject in three brains with a histopathological diagnosis of Alzheimer's disease and post mortem time intervals of less than four hours. By using forty different primer combinations, twenty-eight of thirty-six differentially expressed cDNAs were cloned and sequenced. These cDNAs were further analyzed by reverse Northern blotting (Poirier G. M.-C. et al., Nucleic Acid Res., 25, 913, 1997; Van Gelder R. N. et al., Proc. Natl. Acad. Sci. USA, 87, 1663, 1990) to confirm differential expression between the two AD brain regions. Expression of one of these cDNAs was markedly lower in the inferior temporal lobe than in the sensory-motor cortex. Therefore, the potential importance of this transcript for the selective vulnerability in AD brain has been investigated. The cDNA sequence consisted of 4248 nucleotides and encoded an open reading frame of 516 amino acid residues. Due to a cytidine insertion at nucleotide position 1167, this sequence differed from the much shorter coding region of its homolog KIAA0018 deposited in GenBank (Nomura et al., DNA Res. 1, 27, 1994; GenBank database accession HUMRSC390D13643, 1, 1992; DIMH Human Q15392, 1998). The new gene has been designated SELADIN-1. The homology domain to oxido-reductases are highlighted in red; the homologies to “diminuto like proteins” of other species are underlined. The first 21 amino acid residues represent a putative signal peptide. One possible caspase recognition motif is highlighted in yellow. This putative caspase recognition motif “LEVD” is present within the SELADIN-1 amino acid sequence at position 121-125. In vitro cleavage of SELADIN-1 by caspase 3 or 6 generated four different SELADIN-1 fragments of approximately 50, 40, 30 and 20 kDa, respectively. Secondary structure predictions revealed at least four possible transmembrane domains.

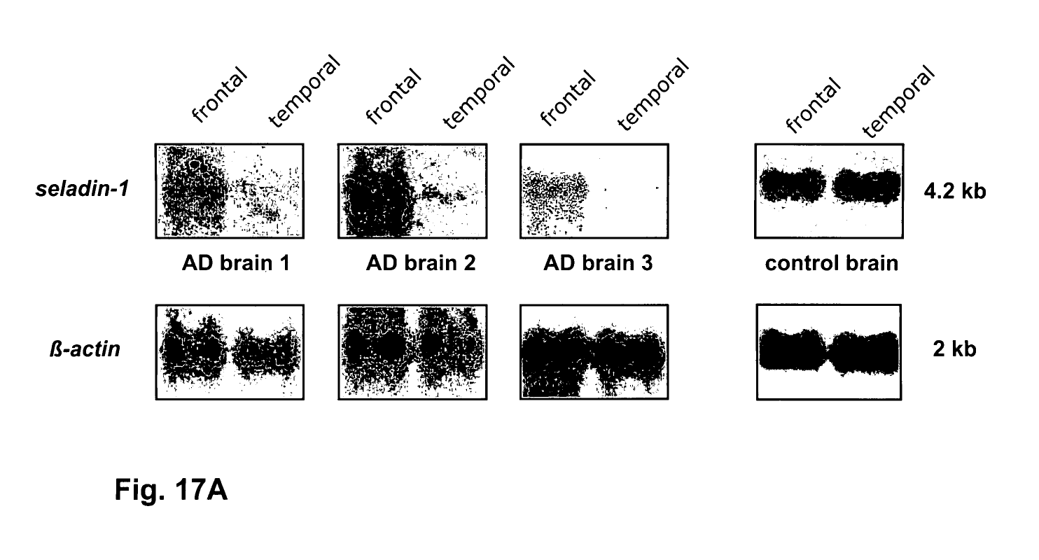

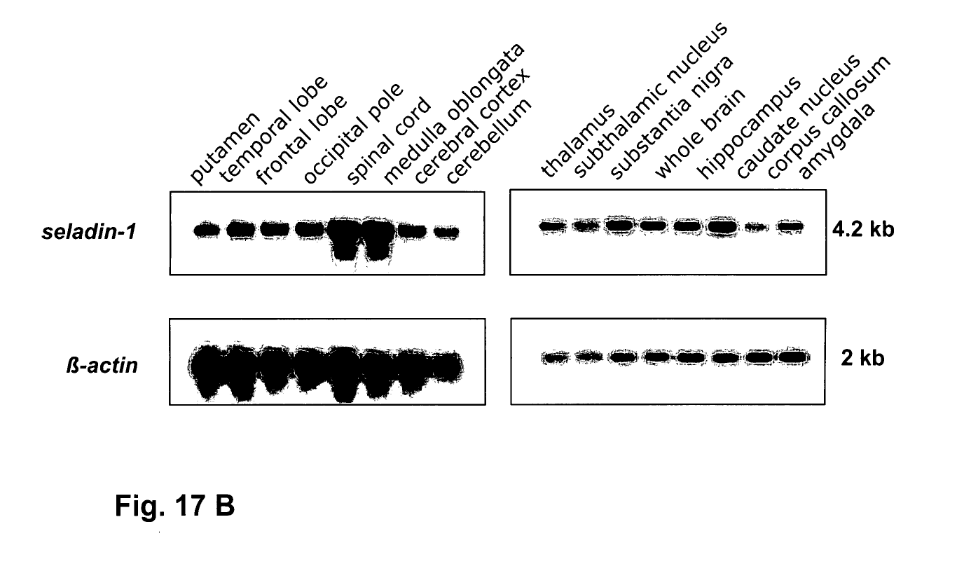

FIG. 17 shows Northern blots of Alzheimer's disease (AD) brain and normal control brain. In AD brains, the expression of SELADIN-1 was substantially lower in the inferior temporal lobe compared to the frontal cortex. In contrast, there was no difference in expression between these two regions in normal control brains (FIGS. 17A, B). Thus, the differential expression of SELADIN-1 between temporal and frontal cortex within individual AD brains initially observed by both differential display and reverse Northerns, was independently confirmed in three other patients. SELADIN-1 is strongly expressed throughout the normal human brain with highest expression in the cortices, in the medulla oblongata and the spinal cord as well as in substantia nigra and the hippocampus (FIG. 17B). A 10 μg of total RNA per lane, extracted with Trizol Reagent (Gibco) from the frontal cortex or the inferior temporal cortex of three different AD brains were separated on a 0.8% formaldehyde-agarose gel and blotted on a Hybond-N+-Nylon Membrane (Amersham). Brain 1: post mortem time interval 3:30 hours, male, 72 years. Brain 2: post mortem time interval 1:30 hours, male, 62 years. Brain 3: post mortem time interval 4 hours, female, 63 years. Control brain: normal brain, post mortem time interval 1:10 hours, female, 80 years. The blots were hybridized with a ³²P-labeled cDNA probe of Seladin-1 from nucleotide 1-3505 and with a ³²P-labeled cDNA control probe of human β-actin as provided by Clontech for the human brain multiple tissue northern blot II and III. B Human brain multiple tissue Northern blot II (Clontech 7755-1) and III (Clontech 7750-1) containing 2 μg of polyA+ RNA per lane from 16 different human brain regions. Blots were hybridized with the same probes as described in A.

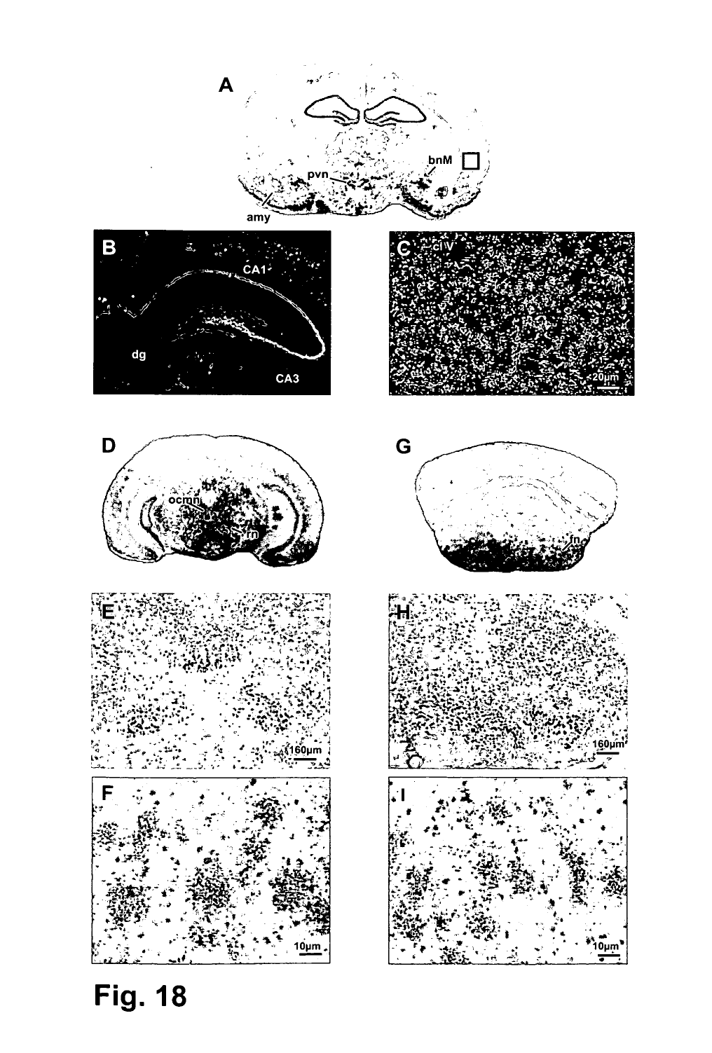

FIG. 18 shows the expression of Seladin-1 in rat brain. in situ hybridization on paraformaldehyde fixed cryostat sections was performed as described by Hartman et al. (Developmental Neuroscience 17, 246, 1995). A 650 bp and a 900 bp fragment of the open reading frame of Seladin-1 were PCR amplified using the following primer pairs:

1s (76-99)

(SEQ ID NO. 3)

5′ GCG CTT ACC GCG CGG CGC CGC ACC 3′
1as (749-726)

(SEQ ID NO. 3)

5′ GAC CAG GGT ACG GCA TAG AAC AGG 3′
3s (803-826)

(SEQ ID NO. 3)

5′ AGA AGT ACG TCA AGC TGC GTT TCG 3′
and
3as (1749-1726)

(SEQ ID NO. 3)

5′ TTC TCT TTG AAA GTG TGG ATC TAG 3′.

PCR fragments were cloned in pGEM-Teasy vector (Promega), cut with EcoRI and cloned in pBluescript KS+. The orientation of the EcoRI cloned fragments was analyzed by PCR. Using the Ambion Maxiscript kit, ³⁵S-UTP labeled antisense and sense riboprobes were generated on NotI and ClaI linearized plasmids with T3 and T7-Polymerase, respectively, according to the manufacturers instructions. Hybridized sections were dipped in NTB-3 photographic emulsion (Kodak), exposed for 5 weeks and counterstained in Mayer's hemalum. A, D, G show photomicrographs of the emulsion dipped sections. pvn paraventricular nucleus, bnM basal nucleus of Meynert, amy amygdala, ocmn oculomotor nucleus, rn red nucleus, fn facial nucleus. B is a dark-field illumination blow up of the hippocampal region. dg dentate gyrus. C is a dark-field illumination blow up of the cortical layer five cl V. E, H show bright field higher magnification photomicrographs of the regions of interest from D and G. F, I DIC (differential interference contrast) illuminations in higher magnification of E and H to demonstrate single neurons stained with silver grains. In rat brain, expression of SELADIN-1 was high in the hippocampal region CA3 (FIG. 18 A, B), in the pyramidal neurons of cortical layer five (FIG. 18 A, C), in the amygdala (FIG. 18 A), in the magnocellular neurons of the basal nucleus of Meynert (FIG. 18 A) and in the reticular zone of the substantia nigra (data not shown). In addition, transcripts were also detected in several brain nuclei including the paraventricular nucleus (FIG. 18 A), the oculomotor nucleus (FIG. 18 D, E), the facial nucleus (FIG. 18 G, F) as well as the red nucleus (FIG. 18 D, E).

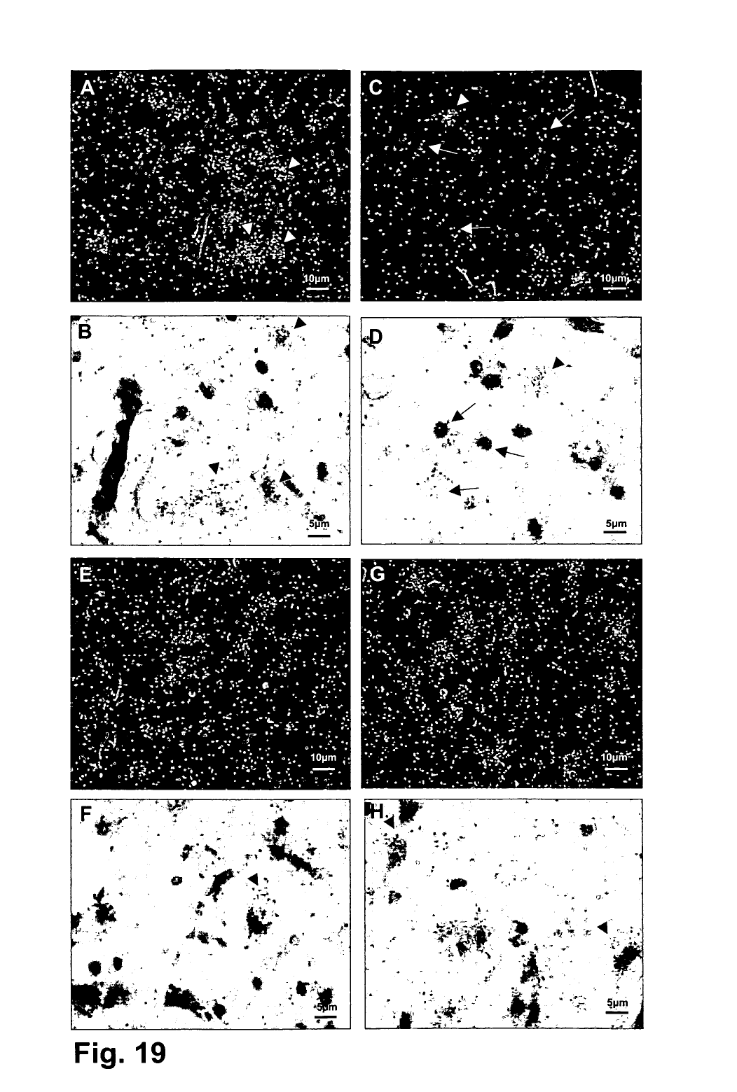

FIG. 19 shows in situ hybridization of human AD (A-D) and normal brain (E-H). in situ hybridization on embedded sections was performed as described (U. Susens, Dev. Neurosci. 19, 410, 1997). The ³⁵S-UTP labeled riboprobe was derived from the first 650 nucleotides of the open reading frame of Seladin-1 cloned in pBluescript KS+ as described in FIG. 18. The hybridized slides were dipped in Kodak NTB-2 emulsion, exposed for 4 weeks. After development, sections were stained with Giemsa. A, C, E and G show dark-field illuminations and B, D, F, H the corresponding bright field photomicrographs. To enhance the visibility of the silver grains in the bright field picture higher magnification is shown. A, B representative hybridization pattern of Seladin-1 in midfrontal cortex of AD brain. C, D representative hybridization pattern of Seladin-1 in superior temporal cortex of AD brain. E, F representative hybridization pattern of Seladin-1 in midfrontal cortex of normal brain. G, H representative hybridization pattern of Seladin-1 in superior temporal cortex of normal brain. Arrowheads indicate neurons packed with silver grains; arrows indicate the neurons with only few grains (D). in situ hybridization of human AD and control brains to study the expression of SELADIN-1 within single neurons, demonstrated that SELADIN-1 mRNA was reduced in the remaining neurons of the temporal cortex in comparison to the neurons in the frontal cortex in the AD brains (FIG. 19, A-D, arrows). In contrast, in normal brains, neuronal expression of SELADIN-1 was identical between the frontal cortex and the temporal cortex (FIG. 19, E-H, arrowheads), confirming the data from differential display and Northern blot analyses. Reduced levels of SELADIN-1 mRNA in the temporal cortex in comparison to the frontal cortex in the AD brain were not only due to cell loss but were also reduced within the remaining neurons.

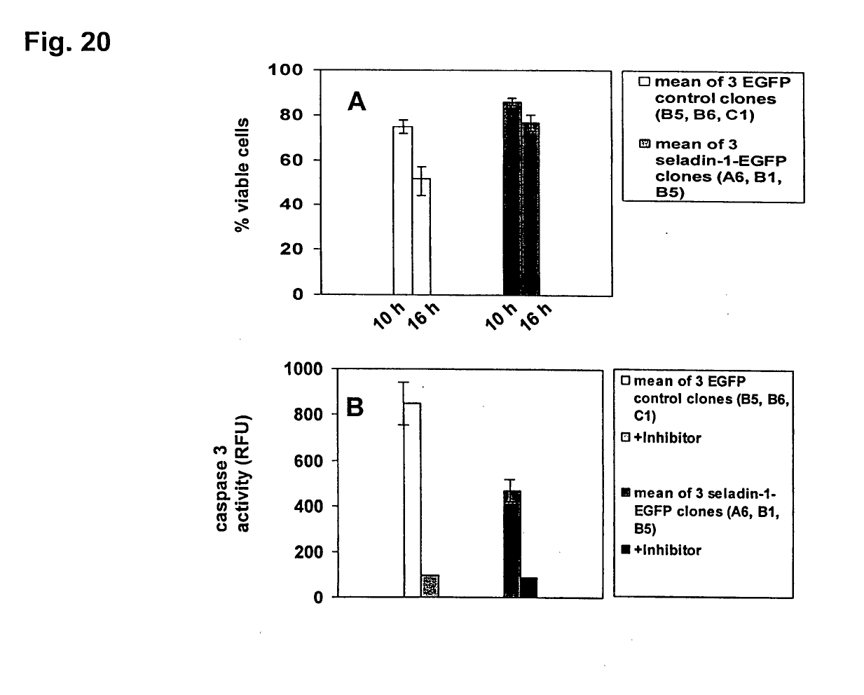

FIG. 20. To analyze SELADIN-1 function as a putative oxido-reductase, human H4 neuroglioma cells were stably transfected with Seladin-1 fused at its C-terminus to EGFP (enhanced green fluorescence protein, Clontech). A 10 and 16 hours after incubation of three seladin-1-EGFP clones and three EGFP-control clones in OptiMEM1 containing 200 μM H₂O₂, cells remaining attached to the culture dish as well as cells in the supernatant were harvested and stained with 7-Amino-actinomycin D (7-ADD) as a standard flow cytometric viability probe to distinguish viable from non viable cells. Only membranes of dead and damaged cells are permeable to this DNA dye and stain positive. Live/dead counts were done on FACSCalibur (Becton Dickinson) counting 10⁵ cells per clone. Means of 2 experiments in triplicate are shown (±SEM). All SELADIN-1 expressing clones tolerated H₂O₂-induced oxidative stress much better than either non-transfected or EGFP expressing clones. After ten hours treatment with 200 μM H₂O₂ nearly 900% of the SELADIN-1 expressing cells and 75-80% of the control cells were viable; sixteen hours after incubation with. 200 μM H₂O₂, however, 80% of the SELADIN-1 expressing cells were still alive whereas only 52% of the control cells were alive at this time point. Untreated control clones revealed a maximum of 5% dead cells at equivalent time intervals. Increased survival rates in SELADIN-1 expressing cells after prolonged exposure to oxidative stress was confirmed by two independent approaches: First, live/dead counts were done on trypan blue stained cells on cell culture dishes and visualized in phase-contrast microscopy in ten randomly chosen fields. Second, nuclei of cells grown on cover slips and fixed with 4% paraformaldehyde were stained with Hoechst dye 33342 (Molecular Probes) and visualized by fluorescence microscopy (data not shown). These measures confirmed that expression of SELADIN-1 conferred resistance against induction of cell death.

B To determine an early marker for apoptotic cell death, the activity of caspase 3 in cell lysates of three SELADIN-1-EGFP clones and three EGFP-control clones was measured using the caspase 3 assay kit from Pharmingen. After induction of apoptosis with 200 μM H₂O₂ for 2 or 4 hours, respectively, cells were washed briefly in PBS and lysed in 10 mM Tris-HCl, pH 7.5, 10 mM NaH₂PO₄4, pH 7.5, 130 mM NaCl, 1% Triton-X-100, 10 nM NaPPi (2 million cells/ml). 50 μl of the cell lysates were incubated in 200 μl HEPES buffer for 1 hour at 37° C. with 5 μg of the caspase 3 fluorogenic substrate Ac-DEBD-CHO in a 96 multiwell plate. The AMC liberated from Ac-DEVD after caspase cleavage was measured on a spectrofluorometer (Spectramax Gemini, Molecular Devices) with an excitation wavelength of 380 nm and an emission wavelength spectrum from 420-460 nm. Means of caspase 3 activity, measured in RFU (relative fluorescence units) of two experiments in triplicates are shown (SEM). Two hours after induction of apoptosis with 200 μM H₂O₂, caspase 3 activity was not detectable in either SELADIN-1-EGFP clones or in the EGFP-control clones. After 4 hours, however, the activity of caspase 3 strongly increased and was found to be approximately two-fold higher in three EGFP-control clones as compared to three SELADIN-1-EGFP clones. This increase in caspase 3 activity was blocked in either condition by the caspase inhibitor Ac-DEVD-CHO.

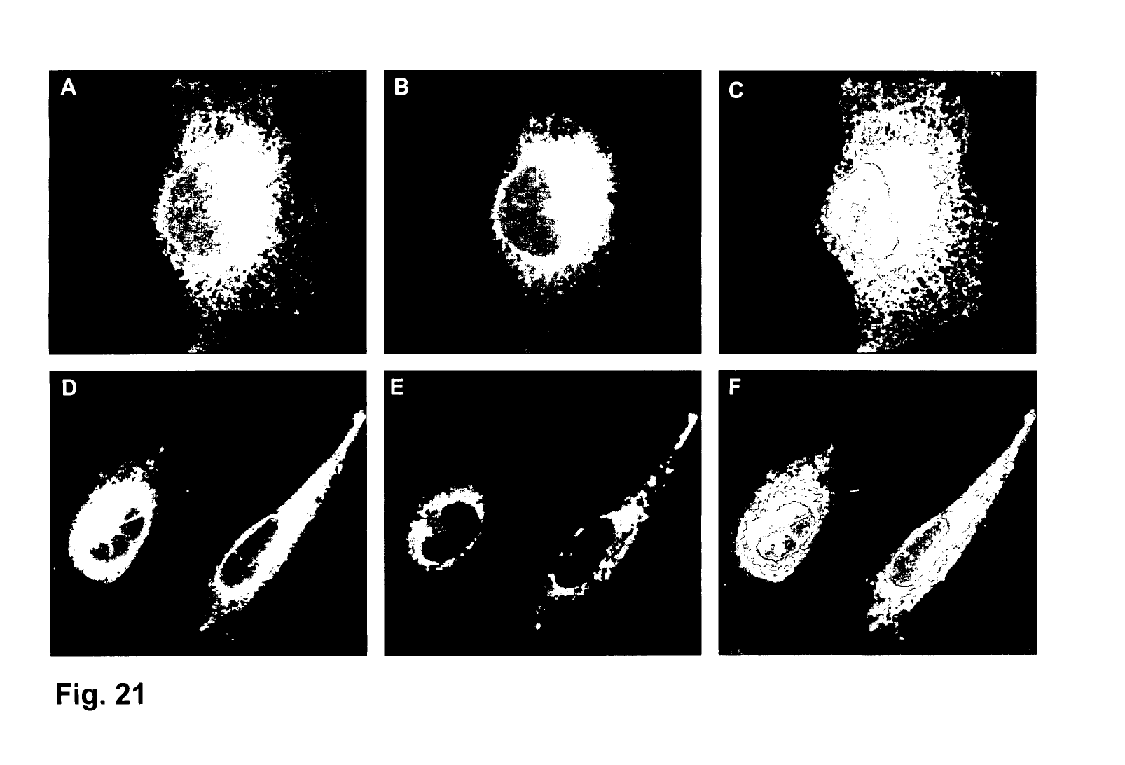

FIG. 21 shows the subcellular localization of SELADIN-1. 114 human neuroglioma cells that stable express a fusion protein of SELADIN-1 with the N-terminus of EGFP (Clontech) were grown on cover slips and fixed in 4% paraformaldehyde in PBS or treated for 45 minutes with 250 nM of the red fluorescent mitochondrial stain MitoTracker red CM H₂Xros (Molecular Probes) before fixation. After fixation cells that have not been prestained with the MitoTracker were permeabilized in 0.2% Triton-X 100 in PBS and blocked over night at 4° C. in 5% low fat milk, 0.1% Triton-X 100 in PBS. Cells were incubated for 2 hours at room temperature with an monoclonal antibody against the mouse anti-protein disulfide isomerase (antiPDImAb, StressGen. Biotechnologies Corp.), a marker for the endoplasmic reticulum, washed and incubated for another hour with an anti-mouse IgG, CY3 labeled secondary antibody (Amersham). Cells were visualized with confocal laser scanning microscopy. A, D Subcellular distribution of the green fluorescent SELADIN-1-EGFP fusion protein. B Staining of the endoplasmatic reticulum with the antiPDImAb and the red fluorescent CY3 labeled secondary antibody. C Overlay from A and B shows the colocalization of SELADIN-1 with the ER-marker, indicated as yellow fluorescence. E Staining of the mitochondria with the red fluorescence MitoTracker CM H₂Xros. F Overlay of D and E. These colocalization studies with markers and antibodies against several subcellular organelles indicated that SELADIN-1-EGFP mainly localized to the endoplasmatic reticulum and not to the mitochondria, despite the presence of a putative mitochondrial localization signal at the N-terminus of SELADIN-1.

Taken together a novel gene SELADIN-1 that has homologies to FAD-dependent oxido-reductases has been identified. It has been shown that it was down-regulated in selectively vulnerable regions of AD brain. in situ hybridization of AD brain sections demonstrated that the reduced mRNA levels are not only due to neuronal loss in affected areas but also reflects reduced mRNA expression of the remaining neurons. Expression of SELADIN-1 in H4 cells conferred resistance to apoptosis by oxidative stress, yet after execution of apoptosis SELADIN-1 is cleaved at putative caspase cleavage sites and therefore is presumably inactivated. These results indicate that SELADIN-1 is an integral component of the cellular machinery protecting cells, in particular neurons, from oxidative stress. Once oxidative stress becomes overwhelming, SELADIN-1 becomes a target for caspase action in the course of apoptosis. SELADIN-1 is a good candidate gene for therapeutical intervention to protect cells against degeneration and cell death. It is in particular, a good candidate gene for therapeutical intervention to protect neurons from Aβinduced cytotoxicity.

### EXAMPLE I

Post-Mortem Alzheimer's Disease Brain Tissues

Brain tissues from Alzheimer's disease patients and control subjects were removed within 6 hours of death, and immediately frozen on dry ice. Parallel sections were fixed in formaldehyde for histopathological confirmation of the diagnosis and for cell counts. Brain areas with massive neuronal cell loss as well as areas with largely preserved neurons were identified for comparisons of gene expression and stored at −80° C. until RNA extractions were performed.

Identification of SELADIN-1 by Differential Display PCR

Total RNA from post-mortem brain tissues was prepared by using the RNeasy kit (Qiagen). The RNA preparations were treated with DNase I (Boehringer Mannheim) together with RNAsin (Promega) for 30 minutes, followed by phenol extraction, and ethanol precipitation. 0.2 mg of each RNA preparation were transcribed to cDNA by using Expand Reverse Transcriptase (Boehringer Mannheim) with one base anchor primers HT₁₁11A, HT₁₁C and HT₁₁G. In the following PCR reaction, the cDNAs were amplified by using HT₁₁A along path the random primers HAP-5 (5′-TGCCGAAGCTTGGAGCTT-3′) and HAP3-T (5′TGCCGAAGCTTTGGTCAT-3′). Taq-polymerase (AmpliTaq, Perkin Elmer Corp.), dGTP, dCTP, and dTTP (Amersham Pharmacia Biotech) and (α³⁵S)-dATP (NEN life science products) were used in a PCR protocol according to Zhao et al. The PCR products were separated on 6% polyacrylamide-urea sequencing gels that were dried subsequently on 3 mm filter paper (Whatman), and X-ray films (Dupont) were exposed for 12 hours.

Cloning and Sequencing

Differential bands were excised from the gel, boiled in water for 10 minutes, centrifuged, and cDNAs were precipitated from the supernatant fluids by using ethanol and glycogen/sodium acetate, followed by dialysis against 10% glycerol for 1 hour through 0.025 mm filters (type VS, Millipore). The dialysates were used as templates for the reamplification reactions that were done under identical conditions as in the differential display PCR, with the exception of the initial cycle for nonspecific annealing. The resulting PCR products were separated by agarose gel electrophoresis, purified from the gel with the QIAEXII Agarose Gel Extraction Kit (Qiagen), and cloned into the Hind III restriction site of pBluescript KS (Stratagene). Cloned cDNA fragments were sequenced with an ABI 377 DNA sequencer (Perkin Elmer Corp.) by using T3 and T7 primers.

Amplification of a SELADIN-1 cDNA-Fragment

A SELADIN-1 cDNA fragment was amplified by using cDNA transcribed from human brain tissue by using RNA High Fidelity Taq-polymerase (Boehringer Mannheim) and SELADIN-1-specific primers for a PCR reaction with 40 cycles of annealing of 70° C. for 1 minute, and polymerization at 72° C. for 3 minutes. The PCR products were separated by agarose gel electrophoresis, purified, and cloned into the Sma I restriction site of pBluescript KS (Stratagene). The cloned PCR product was sequenced, and restriction were used as a probe both for screening a human brain cDNA library and for probing Northern blots.

Northern Blotting

Total RNA from post-mortem human brains were prepared by using the Trizol reagent (Gibco BRL, LifeTechnologies), following the manufacturer's instructions. 5-10 mg of RNA were separated in 1% formaldehyde-containing agarose gels, and the RNA was blotted onto nylon membranes (Hybond-N⁺, Amersham). Membranes were hybridized with (α³²P)-dCTP (NEN) labeled SELADIN-1-specific cDNA probes that were generated by using the Megaprime DNA labeling kit (Amersham). Membranes were washed under high stringency conditions, and X-ray films were exposed for 1 to 72 hours. To control for equal loading of RNA, the identical membranes were probed with a 700 pb cDNA fragment of human glycerolaldehyd-3-phosphate dehydrogenase (GADPH), or with a b-actin cDNA fragment (Clontech).

In Situ Hybridization

Several SELADIN-1-specific cDNA probes of 650 bp and of 900 bp representing the initial two parts of the open reading frame were cloned in pBluescript (Stratagene) and reversely transcribed in the presence of ³⁵S-CTP by using the Ambion transcription kit. in situ hybridization was done with, 14 mm sections of adult rat brain cut on a cryomicrotome, mounted on aminoalkylsilane-treated slides and fixed in 4% paraformaldehyde in PBS for 5 min at room temperature. After washing for 5 min in PBS, sections were acetylated for 10 min, passed through a series of increasing ethanol grades and air dried. Prehybridizations were done in 50% deionized formamide, 25 mM EDTA, 25 mM Pipes, pH 6.8, 0.75 M NaCl, 0.2% SDS, 5× Denhardt's, 10 mM DTT, 250 mg/ml denatured herring sperm DNA and 250 mg/ml yeast tRNA. Hybridization of slides with RNA sense and antisense probes diluted to 2000-5000 cpm/ml in the same buffer with additional 10% dextran sulphate was performed at 50° C. for 12 hours. Slides were then washed four times in 4×SCC for 5 min. each, followed by an incubation for 30 min. at 37° C. with 40 mg/ml RNAseA in 0.5 M NaCl, 10 mM Tris-HCL, pH 7.5, 1 mM EDTA and another 30 min without RNAseA. Then slides were washed twice for 15 min. at 50° C. in 2×SCC and dried through graded ethanols. Slides were exposed to Kodak Biomax x-ray films for 15 days and subsequently dipped in Kodak NTB-3 nuclear track emulsion and exposed for 6 weeks. After developing in Kodak D19 and fixing in Kodak Unifix, slides were counterstained with Mayer” Hemalaun and cover slipped.

Recombinant Expression of SELADIN-1-EGFP Fusion Proteins in Tissue Culture

The complete coding region of SELADIN-1 was subcloned into the N-terminus of the pEGFP-N-1-expression vector (Clontech). Cos-7-cells were transfected with EGFP or with SELADIN-1-EGFP by using the SuperFect transfection reagent from Qiagen according to the manufacturers instructions. Cells were cultured in 3 cm dishes for two days. Part of the cells were stained for the Golgi-apparatus with 0.25 mM BODIPY TR ceramide (molecular probes) for one hour, the other part was treated with 250 nM of the mitochondrial stain Mito Tracker Red CM-H2Xros (Molecular probes) for 45 min. the subcellular localization of the SELADIN-1-EGFP fusion protein was analyzed by confocal laser scanning microscopy using the appropriate filter sets.

1-38. (canceled)

39. An isolated nucleic acid molecule encoding a protein molecule shown in SEQ ID NO: 1.

40. The isolated nucleic acid molecule of claim 39, wherein the nucleic acid molecule is a DNA molecule.

41. The isolated nucleic acid molecule of claim 40, wherein the nucleic acid molecule is a cDNA molecule of the sequence shown in SEQ ID NO: 2.

42. An isolated DNA molecule capable of hybridizing with the complement of the cDNA molecule shown in SEQ ID NO: 2.

43. The isolated DNA molecule of claim 42 encoding a protein molecule, the function of which is to protect cells against degeneration and/or cell death.

44. A vector comprising the isolated nucleic acid molecule according to claim 39.

45. The vector according to claim 44, wherein the vector is a plasmid, a virus, or a bacteriophage.

46. The vector according to claim 45, wherein the vector is a plasmid adapted for expression of the nucleic acid molecule.

47. The vector according to claim 45, wherein the vector is a plasmid containing the regulatory elements necessary for expression of the nucleic acid molecule in a bacterial cell.

48. The vector according to claim 45, wherein the vector is a plasmid containing the regulatory elements necessary for expression of the nucleic acid molecule in a mammalian cell.

49. A host cell transformed with the nucleic acid molecule according to claim 39.

50. The host cell according to claim 49, wherein the cell is a bacterial cell, a yeast cell, a mammalian cell, or an insect cell.
